(19) United States
(12) Patent Application Publication
(10) Pub. No.: US 2008/0044843 A1
(43) Pub. Date: Feb. 21, 2008

(54) BIOMARKERS FOR CHRONIC OBSTRUCTIVE PULMONARY DISEASE

(76) Inventors: Lorah Perlee, Wilton, CT (US); Martin Sorette, Delmar, NY (US); Velizar T. Tchernev, Sofia (BG); Serguel Lejnine, Mercer Island, WA (US); Stephen F. Kingsmore, Santa Fe, NM (US)

Correspondence Address: KIRKPATRICK & LOCKHART PRESTON GATES ELLIS LLP, 1900 MAIN STREET, SUITE 600, IRVINE, CA, 92614-7319, US

(21) Appl. No.: 11/614,893

(22) Filed: Dec. 21, 2006

Related U.S. Application Data
(60) Provisional application No. 60/753,216, filed on Dec. 21, 2005.

(51) Int. Cl. G01N 33/50; C12Q 1/34; C12Q 1/37

(52) U.S. Cl. 435/24; 435/18; 436/87

(57) ABSTRACT

Detection of expression of biomarkers (e.g., protein analytes) whose regulation is perturbed in COPD patients can be used to diagnose COPD, to confirm a diagnosis of COPD, and to assess or prognose progression of COPD. Test substances can be screened for the ability to affect levels of protein analyte expression, thereby identifying potential anti-COPD drugs.

### CROSS REFERENCE TO RELATED APPLICATIONS

The present invention claims the benefit under 37 U.S.C. §119(e) of U.S. Provisional Patent Application No. 60/753,216 filed Dec. 21, 2005, the entire contents of which are incorporated by reference herein.

### FIELD OF THE INVENTION

The invention relates to methods of diagnosing and assessing the progression of chronic obstructive pulmonary disease (COPD). The invention also relates to methods of identifying potential anti-COPD drugs.

### BACKGROUND OF THE INVENTION

Chronic obstructive pulmonary disease (COPD) is a general term used to describe the disorders of emphysema and chronic bronchitis. Emphysema is characterized by an enlargement of air spaces inside the lung. Chronic bronchitis is characterized by excessive mucus production in the bronchial tree. Chronic bronchitis is a clinical definition and denotes those individuals who meet criteria defining the disease. It is not uncommon for an individual to suffer from both disorders.

In 1995, the American Lung Association (ALA) estimated that between 15-16 million Americans suffered from COPD. The ALA estimated that COPD was the fourth-ranking cause of death in the U.S., that the rates of emphysema is 7.6 per thousand population, and the rate for chronic bronchitis is 55.7 per thousand population.

Those inflicted with COPD face disabilities due to the limited pulmonary functions. Usually, individuals afflicted by COPD also face loss in muscle strength and an inability to perform common daily activities. Often, those patients desiring treatment for COPD seek a physician at a point where the disease is advanced. Since the damage to the lungs is irreversible, there is little hope of recovery. Most times, the physician cannot reverse the effects of the disease but can only offer treatment and advice to halt the progression of the disease.

Therefore there exists a need for tests that permit the early detection, risk assessment and monitoring of patients who have or are susceptible to COPD.

### SUMMARY OF THE INVENTION

The invention provides tests that permit the early detection, risk assessment, and monitoring of patients who have or are susceptible to chronic obstructive pulmonary disease (COPD). The tests are based on the identification of protein biomarkers (protein analytes) whose regulation is perturbed in COPD patients. Patterns of differential expression of one or more of these protein analytes (“molecular signatures”) can be used to diagnose COPD, to confirm a diagnosis of COPD, and to assess or prognose progression of COPD. The invention also provides methods of screening test substances to identify potential therapeutic agents which affect levels of protein analyte expression. In addition, the invention identifies biomarkers with differential expression associated with aging and/or gender.

In one embodiment of the present invention, a methods of diagnosing chronic obstructive pulmonary disease in a human subject is provided comprising: comparing a first concentration of at least one analyte in a test sample from the human subject to a second concentration of the at least one analyte in a reference range determined for one or more control samples obtained from one or more human subjects not suffering from chronic obstructive pulmonary disease, wherein the at least one analyte is selected from the group consisting of matrix metalloprotease 9 (MMP9), matrix metalloprotease 10 (MMP-10), eotaxin 2 (Eot2), thymus and activation regulated chemokine (TARC), matrix metalloprotease 7 (MMP7), neutrophil elastase, interleukin 8 (IL-8), macrophage migration inhibitor factor (MIF), interleukin 10 receptor β (IL-10rβ), eotaxin (Eot), matrix metalloprotease 8 (MMP-8), brain-derived neurotrophic factor (BDNF), tissue inhibitor of metalloprotease 1 (TIMP1), amphiregulin (AR), fibroblast growth factor 4 (FGF-4), insulin-like growth factor binding protein 4 (IGFBP-4), tumor necrosis factor receptor 1 (TNF-R1), B lymphocyte chemoattractant (BLC), cutaneous T cell attracting chemokine (CTACK), hemofiltrate CC chemokine 4 (HCC4), interleukin 12p40 (IL-12p40), monocyte chemotactic protein 1 (MCP-1), vascular endothelial growth factor (VEGF), myeloid progenitor inhibitory factor-1 (MPIF-1), hemofiltrate CC chemokine 1 (HCC1), epidermal growth factor (EGF), macrophage inhibitor protein-1b (MIP-1b), and prolactin; and diagnosing chronic obstructive pulmonary disease in the human subject if the first concentration of the at least one analyte is elevated in the test sample relative to the second concentration.

In another embodiment of the present invention, the test sample is blood. In another embodiment, the test sample is serum. In another embodiment, the test sample is plasma. In another embodiment, the test sample is sputum. In another embodiment, the test sample is cerebrospinal fluid.

In an embodiment of the present invention, the one or more human subjects not suffering from chronic obstructive pulmonary disease are non-smokers. In another embodiment, the at least one analyte is MMP-9. In another embodiment, the at least one analyte is MMP-10. In another embodiment, the at least one analyte is Eot-2. In another embodiment, the at least one analyte is TARC. In another embodiment, the at least one analyte is MMP-7. In another embodiment, the at least one analyte is neutrophil elastase. In another embodiment, the at least one analyte is IL-8. In another embodiment, the at least one analyte is MIF. In another embodiment, the at least one analyte is IL-10Rb. In another embodiment, the at least one analyte is Eot. In another embodiment, the at least one analyte is MMP-8. In another embodiment, the at least one analyte is BDNF. In another embodiment, the at least one analyte is TIMP-1. In another embodiment, the at least one analyte is AR. In another embodiment,

In an embodiment of the present invention, the one or more human subjects not suffering from chronic obstructive pulmonary disease are smokers. In another embodiment, the at least one analyte is IGF-II. In another embodiment, the at least one analyte is IGFBP-3. In another embodiment, the at least one analyte is neutrophil elastase. In another embodiment, the at least one analyte is prolactin.

In one embodiment of the present invention, a method of distinguishing exacerbators in chronic obstructive pulmonary disease from non-exacerbators is provided, the method comprising: comparing a first concentration of at least one analyte in a test sample from an exacerbator human subject to a second concentration of the at least one analyte in a reference range determined from one or more samples obtained from one or more non-exacerbator human subjects suffering from chronic obstructive pulmonary disease wherein the at least one analyte is selected from the group consisting of BLC, HGF, and MIP-1delta, wherein the first concentration of the at least one analyte is elevated relative to the second concentration.

In another embodiment of the present invention, the test sample is blood. In another embodiment, the test sample is serum. In another embodiment, the test sample is plasma. In another embodiment, the test sample is sputum. In another embodiment, the test sample is cerebrospinal fluid.

In an embodiment of the present invention, the exacerbators undergo at least 3 exacerbation events per year. In another embodiment, the at least one analyte is BLC. In another embodiment, the at least one analyte is HGF. In another embodiment, the at least one analyte is MIP-1delta.

In one embodiment of the present invention, a method of distinguishing infrequent exacerbators in chronic obstructive pulmonary disease from non-exacerbators is provided, the method comprising: comparing concentration of at least one analyte in a test sample from said infrequent exacerbator human subject to concentration of said at least one analyte in a reference range that was determined for one or more samples obtained from one or more non-exacerbator human subjects suffering from chronic obstructive pulmonary disease wherein the at least one analyte is selected from the group consisting of BDNF, CRP and Mip-1 beta, wherein the concentration of the at least one analyte is elevated in the test sample relative to the reference range.

In another embodiment of the present invention, the test sample is blood. In another embodiment, the test sample is serum. In another embodiment, the test sample is plasma. In another embodiment, the test sample is sputum. In another embodiment, the test sample is cerebrospinal fluid.

In an embodiment of the present invention, the infrequent exacerbators undergo 1 to 2 exacerbation events per year. In another embodiment, the at least one analyte is BDNF. In another embodiment, the at least one analyte is CRP. In another embodiment, the at least one analyte is Mip-1 beta.

In one embodiment of the present invention, a method of distinguishing infrequent exacerbators in chronic obstructive pulmonary disease from non-exacerbators is provided, the method comprising: comparing a first concentration of at least one analyte in a test sample from the infrequent exacerbator human subject to concentration of the at least one analyte in a reference range determined for one or more samples obtained from one or more non-exacerbator human subjects suffering from chronic obstructive pulmonary disease wherein the at least one analyte is selected from the group consisting of IL-2sR alpha and PF-4; wherein the concentration of the at least one analyte is depressed in the test sample relative to the reference range.

In another embodiment of the present invention, the test sample is blood. In another embodiment, the test sample is serum. In another embodiment, the test sample is plasma. In another embodiment, the test sample is sputum. In another embodiment, the test sample is cerebrospinal fluid.

In an embodiment of the present invention, the infrequent exacerbators undergo 1 to 2 exacerbation events per year. In another embodiment, the at least one analyte is IL-2sR. In another embodiment, the at least one analyte is PF-4.

In one embodiment of the present invention, a method of distinguishing exacerbators in chronic obstructive pulmonary disease from infrequent exacerbators is provided, the method comprising: comparing concentration of at least one analyte in a test sample from said exacerbator human subject to concentration of said at least one analyte in a reference range that was determined for one or more samples obtained from one or more infrequent exacerbator human subjects suffering from chronic obstructive pulmonary disease wherein the at least one analyte is selected from the group consisting of: BDNF, FGF-2, Flt3Lig, MIF, MIP-1 delta, and NT-4, wherein the concentration of the at least one analyte is elevated in the test sample relative to the reference range.

In another embodiment of the present invention, the test sample is blood. In another embodiment, the test sample is serum. In another embodiment, the test sample is plasma. In another embodiment, the test sample is sputum. In another embodiment, the test sample is cerebrospinal fluid.

In an embodiment of the present invention, the infrequent exacerbators undergo 1 to 2 exacerbation events and exacerbators undergo 3 or more events per year. In another embodiment, the at least one analyte is BDNF. In another embodiment, the at least one analyte is FGF-2. In another embodiment, the at least one analyte is Flt3Lig. In another embodiment, the at least one analyte is MIF. In another embodiment, the at least one analyte is MIP-1 delta. In another embodiment, the at least one analyte is NT-4.

In one embodiment of the present invention, a method of identifying biomarkers for smoking is provided, the method comprising: comparing concentration of at least one analyte in a test sample from a smoker human subject to concentration of said at least one analyte in a reference range that was determined for one or more samples obtained from one or more non-smoker human subjects wherein the at least one analyte is selected from the group consisting of: CD 141, ENA-78, ICAM-1, leptin, prolactin, TARC, TIMP-2, AR, BDNF, BLC, Eot-2, IL-10Rb, IL-10p40, MCP-1, MIF, MMP-8, MMP-9, MMP-10, neutrophil elastase, prolactin, TARC, TIMP-1, and VEGF, wherein the concentration of the at least one analyte is altered in the test sample relative to the reference range.

In another embodiment of the present invention, the test sample is blood. In another embodiment, the test sample is serum. In another embodiment, the test sample is plasma. In another embodiment, the test sample is sputum. In another embodiment, the test sample is cerebrospinal fluid.

In another embodiment, the at least one analyte is CD 141. In another embodiment, the at least one analyte is ENA-78. In another embodiment, the at least one analyte is ICAM-1. In another embodiment, the at least one analyte is leptin. In another embodiment, the at least one analyte is prolactin. In another embodiment, the at least one analyte is TARC. In another embodiment, the at least one analyte is TIMP-2.

In an embodiment of the present invention, the concentration of the at least one analyte is elevated in the test sample relative to the reference range. In another embodiment, the concentration of the at least one analyte is depressed in the test sample relative to the reference range. In another embodiment, the smoker human subjects suffer from chronic obstructive pulmonary disease. In another embodiment, the altered analyte comprises AR. In another embodiment, the altered analyte comprises BDNF. In another embodiment, the altered analyte comprises BLC. In another embodiment, the altered analyte comprises Eot-2. In another embodiment, the altered analyte comprises IL-10Rb. In another embodiment, the altered analyte comprises IL-10p40. In another embodiment, the altered analyte comprises MCP-1. In another embodiment, the altered analyte comprises MIF. In another embodiment, the altered analyte comprises MMP-8. In another embodiment, the altered analyte comprises MMP-9. In another embodiment, the altered analyte comprises MMP-10. In another embodiment, the altered analyte comprises neutrophil elastase. In another embodiment, the altered analyte comprises prolactin. In another embodiment, the altered analyte comprises TARC. In another embodiment, the said altered analyte comprises TIMP-1. In another embodiment, the altered analyte comprises VEGF.

In one embodiment of the present invention, chronic obstructive pulmonary disease is diagnosed if at least two of the analytes in the test sample are elevated. In another embodiment, chronic obstructive pulmonary disease is diagnosed if at least three of the analytes in the test sample are elevated. In another embodiment, chronic obstructive pulmonary disease is diagnosed if at least four of the analytes in the test sample are elevated. In another embodiment, chronic obstructive pulmonary disease is diagnosed if at least five of the analytes in the test sample are elevated. In another embodiment, chronic obstructive pulmonary disease is diagnosed if at least six of the analytes in the test sample are elevated.

In one embodiment of the present invention, the step of selecting analytes from a group of analytes comprises selecting from a group consisting of: MMP9, MMP-10, Eot2, TARC, MMP7, Neut Elast, IL-8, MIF, IL-10rb, Eot, MMP-8, BDNF, TIMP1, AR, FGF-4, IGFBP-4, TNF-R1, BLC, CTACK, HCC4, IL-12p40, MCP-1, VEGF, MPIF-1, HCC1, EGF, MIP-1b, Prolactin, IL-2sRa, ProteinC, LT bR, IGF-IR, IL-17, MIG, IL-3, ICAM-1, GM-CSF, IL-1srII, ENA-78, MIP-1d, PARC, Rantes, IGF-II, NT3, NT4, AgRP, ALCAM, IGFBP-3, IGFBP-6, CD40, Flt3Lig, HCG, VAP-1, Follistatin, MIP3b, PAI-II, PECAM1, ProteinS, TRAIL R4, and PF4.

In one embodiment of the present invention, a method of diagnosing chronic obstructive pulmonary disease in a human subject suffering from exacerbated chronic obstructive pulmonary disease is provided, the method comprising: comparing concentration of at least one analyte in a test sample from said human subject to concentration of said at least one analyte in a reference range that was determined for one or more quiescent samples obtained from one or more human subjects not suffering from chronic obstructive pulmonary disease wherein the at least one analyte is selected from the group consisting of: GRO-beta, ICAM-3, TIMP-1, ENA-78, Flt3Lig, IL-13, IL-15, IL-3, IL-4, MIP-1 delta, NT3, NT4, PARC, TARC, sgp130, and IGFBP-3; and diagnosing chronic obstructive pulmonary disease in the human subject if the concentration of the at least one analyte is altered in the test sample relative to the reference range.

In another embodiment of the present invention, the test sample is blood. In another embodiment, the test sample is serum. In another embodiment, the test sample is plasma. In another embodiment, the test sample is sputum. In another embodiment, the test sample is cerebrospinal fluid.

In another embodiment, the concentration of the at least one analyte is elevated in the test sample relative to the reference range. In another embodiment, the at least one analyte is GRO-beta. In another embodiment, the at least one analyte is ICAM-3. In another embodiment, the at least one analyte is TIMP-1.

In another embodiment, the concentration of the at least one analyte is depressed in the test sample relative to the reference range. In another embodiment, the at least one analyte is ENA-78. In another embodiment, the at least one analyte is Flt3Lig. In another embodiment, the at least one analyte is IL-13. In another embodiment, the at least one analyte is IL-15. In another embodiment, the at least one analyte is IL-3. In another embodiment, the at least one analyte is IL-4. In another embodiment, the at least one analyte is MIP-1 delta. In another embodiment, the at least one analyte is NT3. In another embodiment, the at least one analyte is NT4. In another embodiment, the at least one analyte is PARC. In another embodiment, the at least one analyte is TARC. In another embodiment, the at least one analyte is sgp130. In another embodiment, the at least one analyte is IGFBP-3.

In an embodiment of the present invention, chronic obstructive pulmonary disease is diagnosed if concentrations of at least two of the analytes in the test sample are altered. In another embodiment, chronic obstructive pulmonary disease is diagnosed if concentrations of at least three of the analytes in the test sample are altered. In another embodiment, chronic obstructive pulmonary disease is diagnosed if concentrations of at least four of the analytes in the test sample are altered. In another embodiment, chronic obstructive pulmonary disease is diagnosed if concentrations of at least five of the analytes in the test sample are altered. In another embodiment, chronic obstructive pulmonary disease is diagnosed if concentrations of at least six of the analytes in the test sample are altered.

In one embodiment of the present invention, a method of diagnosing chronic obstructive pulmonary disease in a patient is provided comprising: comparing a first concentration of prolactin in a test sample from the patient to a second concentration of prolactin in a reference range determined from one or more control samples obtained from one or more human subjects not suffering from chronic obstructive pulmonary disease; and diagnosing chronic obstructive pulmonary disease in the patient if the first concentration of prolactin is elevated in the test sample relative to the second concentration. In another embodiment, the test sample is selected from the group consisting of serum, sputum, blood, plasma, and cerebrospinal fluid. In another embodiment, the one or more human subjects not suffering from chronic obstructive pulmonary disease are smokers and the method further comprises: comparing a first concentration of at least one analyte selected from the group consisting of IGF-II and IGFBP-3 in the test sample to a second concentration of the analyte in a reference range determined from one or more control samples obtained from said human subjects; and diagnosing chronic obstructive pulmonary disease in the patient if the first concentration of the at least one analyte is elevated in the test sample relative to the second concentrations.

In another embodiment of the present invention, the method further comprises comparing a first concentration of neutrophil elastase in the test sample to a second concentration of neutrophil elatase in a reference range determined from one or more control samples obtained from one or more human subjects not suffering from chronic obstructive pulmonary disease; and diagnosing chronic obstructive pulmonary disease in the patient if the first concentration of prolactin and the first concentration of neutrophil elastase are elevated in the test sample relative to the second concentrations. In another embodiment, the one or more human subjects not suffering from chronic obstructive pulmonary disease are smokers and the method further comprises: comparing a first concentration of at least one analyte selected from the group consisting of insulin-like growth factor II (IGF-II) and insulin-like growth factor binding protein 3 (IGFBP-3), in the test sample to a second concentration of the analyte in a reference range determined from one or more control samples obtained from said human subjects; and diagnosing chronic obstructive pulmonary disease in the patient if the first concentration of the at least one analyte is elevated in the test sample relative to the second concentrations.

In one embodiment of the present invention, a method of diagnosing chronic obstructive pulmonary disease in a patient is provided comprising: comparing a first concentration of at least one analyte in a test sample from the patient to a second concentration of the at least one analyte in a reference range determined from one or more control samples obtained from one or more human subjects not suffering from chronic obstructive pulmonary disease, wherein the at least one analyte is selected from the group consisting of matrix metalloprotease 9 (MMP-9), matrix metalloprotease 10 (MMP-10), eotaxin 2 (Eot-2), thymus and activation regulated chemokine (TARC), matrix metalloprotease 7 (MMP-7), neutrophil elastase, interleukin 8 (IL-8), macrophage migration inhibitor factor (MIF), interleukin 10 receptor (IL-10Rβ), eotaxin, matrix metalloprotease 8 (MMP-8), brain-derived neurotrophic factor (BDNF), tissue inhibitor of metalloprotease 1 (TIMP-1), amphiregulin, fibroblast growth factor 4 (FGF-4), insulin-like growth factor binding protein 4 (IGFBP-4), tumor necrosis factor receptor 1 (TNF-RI), B lymphocyte chemoattractant (BLC), cutaneous T cell attracting chemokine (CTACK), hemofiltrate CC chemokine 4 (HCC4), interleukin 12p40 (IL-12p40), monocyte chemotactic protein 1 (MCP-1), vascular endothelial growth factor (VEGF), myeloid progenitor inhibitory factor-1 (MPIF-1), hemofiltrate CC chemokine 1 (HCC1), epidermal growth factor (EGF), macrophage inhibitor protein-Ib (MIP-1b), and prolactin; and diagnosing chronic obstructive pulmonary disease in the patient if the first concentration of the at least one analyte is elevated in the test sample relative to the second concentration.

In another embodiment, the one or more human subjects not suffering from chronic obstructive pulmonary disease are non-smokers. In another embodiment, the at least one analyte is MMP-9. In another embodiment, the at least one analyte is MMP-10. In another embodiment, the at least one analyte is Eot-2. In another embodiment, the at least one analyte is TARC. In another embodiment, the at least one analyte is MMP-7. In another embodiment, the at least one analyte is IL-8. In another embodiment, the at least one analyte is MIF. In another embodiment, the at least one analyte is IL-10Rβ. In another embodiment, the at least one analyte is eotaxin. In another embodiment, the at least one analyte is MMP-8. In another embodiment, the at least one analyte is BDNF. In another embodiment, the at least one analyte is TIMP-1. In another embodiment, the at least one analyte is amphiregulin. In another embodiment, the at least one analyte is neutrophil elastase.

In one embodiment of the present invention, a method of diagnosing chronic obstructive pulmonary disease in a patient is provided comprising: comparing a first concentration of neutrophil elastase in a test sample from the patient to a second concentration of neutrophil elastase in a reference range determined from one or more control samples obtained from one or more human subjects not suffering from chronic obstructive pulmonary disease and wherein the one or more human subjects not suffering from chronic obstructive pulmonary disease are smokers, and diagnosing chronic obstructive pulmonary disease in the patient if the first concentration of neutrophil elastase is elevated in the test sample relative to the second concentration. In another embodiment, the method further comprises comparing a first concentration of at least one analyte selected from the group consisting of IGF-II and IGFBP-3 in the test sample to a second concentration of the analyte in a reference range determined from one or more control samples obtained from the human subjects; and diagnosing chronic obstructive pulmonary disease in the patient if the first concentration of the at least one analyte is elevated in the test sample relative to the second concentrations.

In one embodiment of the present invention, a method of distinguishing exacerbator patients in chronic obstructive pulmonary disease from non-exacerbator patients is provided, the method comprising: comparing a first concentration of at least one analyte in a test sample from the exacerbator patient to a second concentration of the at least one analyte in a reference range determined from one or more samples obtained from one or more non-exacerbator patients suffering from chronic obstructive pulmonary disease, wherein the at least one analyte is selected from a group consisting of BLC, hepatocyte growth factor (HGF), and macrophage inhibitor protein-I delta (MIP-1 delta), and wherein the first concentration of the at least one analyte is elevated relative to the second concentration. In another embodiment, the test sample is selected from the group consisting of serum sputum, blood, plasma, and cerebrospinal fluid. In another embodiment, the at least one analyte is BLC. In another embodiment, the at least one analyte is HGF. In another embodiment, the at least one analyte is MIP-1 delta.

In one embodiment of the present invention, a method of diagnosing chronic obstructive pulmonary disease in a patient is provided comprising: assaying in a test sample from the patient a panel having two or more analytes by comparing a first concentration of each analyte in the panel to a second concentration of each analyte in the panel wherein the second concentration comprises a reference range determined from one or more control samples obtained from one or more human subjects not suffering from chronic obstructive pulmonary disease, and diagnosing chronic obstructive pulmonary disease in the patient if the first concentrations of the two or more analytes are elevated in the test sample relative to the second concentrations, wherein the panel comprises at least one matrix metalloprotease selected from the group consisting of matrix metalloprotease 7 (MMP-7), matrix metalloprotease 8 (MMP-8), matrix metalloprotease 9 (MMP-9), and matrix metalloprotease 10 (MMP-10) and at least one analyte selected from the group consisting of Eot-2, TARC, neutrophil elastase, BDNF, IL-8, TIMP-1, and amphiregulin. In another embodiment, the at least one analyte is Eot-2. In another embodiment, the at least one analyte is TARC. In another embodiment, the at least one analyte is neutrophil elastase. In another embodiment, the at least one analyte is BDNF. In another embodiment, the at least one analyte is IL-8. In another embodiment, the at least one analyte is TIMP-1. In another embodiment, the at least one analyte is amphiregulin.

### BRIEF DESCRIPTION OF THE DRAWINGS

FIG. 1. Plots of log2 transformed MFI (y-axis) of analytes neutrophil elastase (A), prolactin (B), IGF-II (C) and IGFBP-3 (D) with significant differences observed between COPD and smoking controls. X-axis represents healthy control (CTRL) and different COPD exacerbator categories.

FIG. 2. Plots of log2 transformed MFI (Y-axis) of analytes BLC (A), HGF (B), and MIP-1d (C) with significant differences observed between COPD non-exacerbators and frequent exacerbators. X-axis represents healthy control (CTRL) and different COPD exacerbator groupings. Error bars represent standard deviation.

FIG. 3. Plots of log2 transformed MFI (Y-axis) of analytes BDNF (A), CRP (B), IL-2sRa (C), MIP-1d (D) and PF-4 (E) with significant differences observed between COPD non-exacerbators and infrequent exacerbators. X-axis represents healthy control (CTRL) and different COPD exacerbator groupings. Error bars represent standard deviation.

FIG. 4. Plot of log2 transformed MFI (Y-axis) of analytes BDNF (A), Flt3Lig (B), MIF (C), MIP-1d (D), NT4 (E) and FGF-2 (F) with significant differences observed between COPD infrequent and frequent exacerbators. X-axis=healthy control (CTRL) and different COPD exacerbator groupings. Error bars represent standard deviation.

FIG. 5. TNF-R1 smoking*COPD interaction plot. Y-axis represents log2 MFI. X-axis represents healthy control (CTRL) and COPD.

FIG. 6. Schematic representation of a sample protein microarray slide with 16 subarrays. “Subarray” refers to the 16 wells, or circular analysis sites, on the slide. “Array” refers to the antibody content printed in a well. Each microarray slide contains only one type of array.

FIG. 7. Distributions of CVs between different slides for arrays 1-4 (FIGS. 7A-D).

FIG. 8. Analyte levels in sputum samples for array 1. Y-axis represents log2 of MSI, X-axis represents baseline and exacerbated samples for each analyte shown above the graph.

FIG. 9. Analyte levels in sputum samples for array 2. Y-axis represents log2 of MSI, X-axis represents baseline and exacerbated samples for each analyte shown above the graph.

FIG. 10. Analyte levels in sputum samples for array 3. Y-axis represents log2 of MSI, X-axis represents baseline and exacerbated samples for each analyte shown above the graph.

FIG. 11. Analyte levels in sputum samples for array 4. Y-axis represents log2 of MSI, X-axis represents baseline and exacerbated samples for each analyte shown above the graph.

FIG. 12. Scatter plot of log2 (M.F.I.) within patient between conditions (quiescent and exacerbated).

FIG. 13. Mean (A) and individual patient (B) intensity levels for analyte GRO beta.

FIG. 14. Mean (A) and individual patient (B) intensity levels for analyte ICAM-3.

FIG. 15. Mean (A) and individual patient (B) intensity levels for analyte TIMP-1.

FIG. 16. Mean (A) and individual patient (B) intensity levels for analyte ENA-78.

FIG. 17. Mean (A) and individual patient (B) intensity levels for analyte Flt3Lig.

FIG. 18. Mean (A) and individual patient (B) intensity levels for analyte IL-13.

FIG. 19. Mean (A) and individual patient (B) intensity levels for analyte IL-15.

FIG. 20. Mean (A) and individual patient (B) intensity levels for analyte IL-3.

FIG. 21. Mean (A) and individual patient (B) intensity levels for analyte IL-4.

FIG. 22. Mean (A) and individual patient (B) intensity levels for analyte MIP-1 delta.

FIG. 23. Mean (A) and individual patient (B) intensity levels for analyte NT-3.

FIG. 24. Mean (A) and individual patient (B) intensity levels for analyte NT-4.

FIG. 25. Mean (A) and individual patient (B) intensity levels for analyte PARC.

FIG. 26. Mean (A) and individual patient (B) intensity levels for analyte TARC.

FIG. 27. Mean (A) and individual patient (B) intensity levels for analyte sgp130.

FIG. 28. Mean (A) and individual patient (B) intensity levels for analyte IGFBP-3.

FIG. 29. Analysis of variance for 16 analytes found to be significantly different between exacerbation and baseline samples. Y-axis represents percent variance, X-axis represents analyte name.

FIG. 30. Dose-response curves for IL-6 in dilution buffer, plasma and serum, based on all data. Each data point represents average from 2 assay wells. Error bars represent standard deviations of the replicate well means. Corresponding linear-log (A) and log-log (B) graphs are shown.

FIG. 31. Dose-response curves for IL-8 in dilution buffer, plasma and serum, based on all data. Each data point represents average from 2 assay wells. Error bars represent standard deviations of the replicate well means. Corresponding linear-log (A) and log-log (B) graphs are shown.

FIG. 32. Dose-response curves for IL-2 in dilution buffer, plasma and serum, based on all data. Each data point represents average from 2 assay wells. Error bars represent standard deviations of the replicate well means. Corresponding linear-log (A) and log-log (B) graphs are shown.

FIG. 33. Dose-response curves for TNF-α in dilution buffer, plasma and serum, based on all data. Each data point represents average from 2 assay wells. Error bars represent standard deviations of the replicate well means. Corresponding linear-log (A) and log-log (B) graphs are shown.

### DETAILED DESCRIPTION OF THE INVENTION

The invention provides tests that permit the early detection, risk assessment, and monitoring of patients who have or are susceptible to chronic obstructive pulmonary disease (COPD). The tests are based on the identification of protein biomarkers (protein analytes) whose regulation is perturbed in COPD patients. Patterns of differential expression of one or more of these protein analytes (“molecular signatures”) can be used to diagnose COPD, to confirm a diagnosis of COPD, and to assess or prognose progression of COPD. The invention also provides methods of screening test substances to identify potential therapeutic agents which affect levels of protein analyte expression. In addition, the invention identifies biomarkers with differential expression associated with aging and/or gender.

The protein analytes disclosed in the examples below were identified using SAS® MIXED procedure (SAS Institute Inc., 1992, SAS Technical Report P-229, SAS/STAT Software: Changes and Enhancements, Release 6.07, Cary, N.C.: SAS Institute Inc.), which was applied to determine significant changes in protein analyte expression with age and OA. Several statistical models were used to test the association of protein analyte levels with age and with diagnosis. As set forth in more detail in the examples, below, 19 protein analytes differed significantly over time (p≦0.05) in expression between OA patients and healthy controls. Expression of some of these protein analytes was significantly different for more than one effect.

Serum

The GSK 8 COPD study was designed to identify biomarkers that were differentially expressed in COPD patients, smokers, and non-COPD healthy controls. Serum samples from 48 COPD patients and 48 healthy controls were provided. There were 8 active smokers in each group. The COPD patients were divided into three categories consisting of individuals with 0, 1-2, or greater than 2 exacerbation events, with 16 patients in each category. One sample (from a patient with two or more exacerbation events) did not pass QC, and was not included in the analysis. The specific goals and the corresponding analyses are summarized below.
1. Differentiate COPD subjects from both smoking and non-smoking controls using serum analyte profiles and identify biomarkers for COPD.
   a. All COPD patients (n=47) and all non-smoking healthy controls (n=40) were compared. 60 serum analytes were elevated in COPD patients, with p-values below 0.05. Some of the largest differences were seen for MMP-9, MMP-10, Eot-2, TARC, MMP-7, neutrophil elastase, IL-8, MIF, IL-10Rb, Eot, MMP-8, BDNF, TIMP-1, and AR.
   b. All COPD (n=47) and healthy smokers (n=8) were compared. Four analytes were elevated in COPD with p-values below 0.05, IGF-II, IGFBP-3, neutrophil elastase and prolactin.

Neutrophil elastase and prolactin were identified in both analyses with effect size greater than 0.6, which fits the profile for specific COPD biomarkers.
2. Differentiate between frequent COPD exacerbators (>2 per year), infrequent exacerbators (>0<=2 per year), and non-exacerbators (0 per year) using a serum analyte profile.
   a. COPD non-exacerbators (n=16) and COPD frequent exacerbators (n=15) were compared. Three analytes with p-value less than 0.05 were identified, BLC, HGF, and MIP-1δ. All were elevated in frequent exacerbators.
   b. COPD non-exacerbators were compared with infrequent exacerbators (n=16). BDNF, CRP, and MIP-1β all showed increased expression in non-exacerbators. IL-2sRα and PF-4 were decreased in non-exacerbators. Levels of PF-4 were near saturation and its significance should be interpreted with caution.
   c. COPD frequent exacerbators were compared with infrequent exacerbators. BDNF, FGF-2, Flt3Lig, MIF, MIP-1□, and NT-4 were all upregulated in frequent exacerbators.

MIP-1δ levels were increased in the most frequent exacerbators compared to the less frequent exacerbators. BDNF levels varied with exacerbation frequency in a non-linear manner.
3. Identify potential changes in analyte profile that can be attributed to smoking.
   a. All smokers (n=16) and all non-smokers (n=79) were compared. Seven analytes with significant differences (p value less than 0.05) were identified: CD 141, ENA-78, ICAM-1, leptin, prolactin, TARC and TIMP-2.

Comparisons were also performed using 2-way analysis of variance (2-way ANOVA). This analysis can control for potential confounding effects in the one-way comparisons listed above, for instance the effect of smoking on COPD comparisons. This analysis also identified markers for:
COPD: examples include AR, BDNF, BLC, Eot-2, IL-10Rb, IL-10p40, MCP-1, MIF, MMP-10, MMP-8, MMP-9, neutrophil elastase, prolactin, TARC, TIMP-1, VEGF, all of which overlap with the COPD markers identified in the one-way analysis above.
Smoking: CD141, ENA-78, ICAM-1, leptin, MMP-10, prolactin, TARC and TIMP-2, all of which, except MMP-10, overlap with the markers for smoking identified in the one-way analysis.

Identifying overlapping sets of markers using two different statistical methods demonstrates that the changes observed in this sample set are robust. The relatively small number of individuals in some of the categories limited the statistical power of the analysis in some cases, but some of the larger changes in protein expression associated with COPD and smoking were significant.

Sputum

The goals of the GSK COPD Study were to evaluate the compatibility of sputum samples with MSI's protein microarrays and to profile respiratory system cytokine levels modulated during exacerbation of COPD. A total of 10 sputum samples were tested: an exacerbated COPD and a quiescent sample (baseline) from each of five patients.

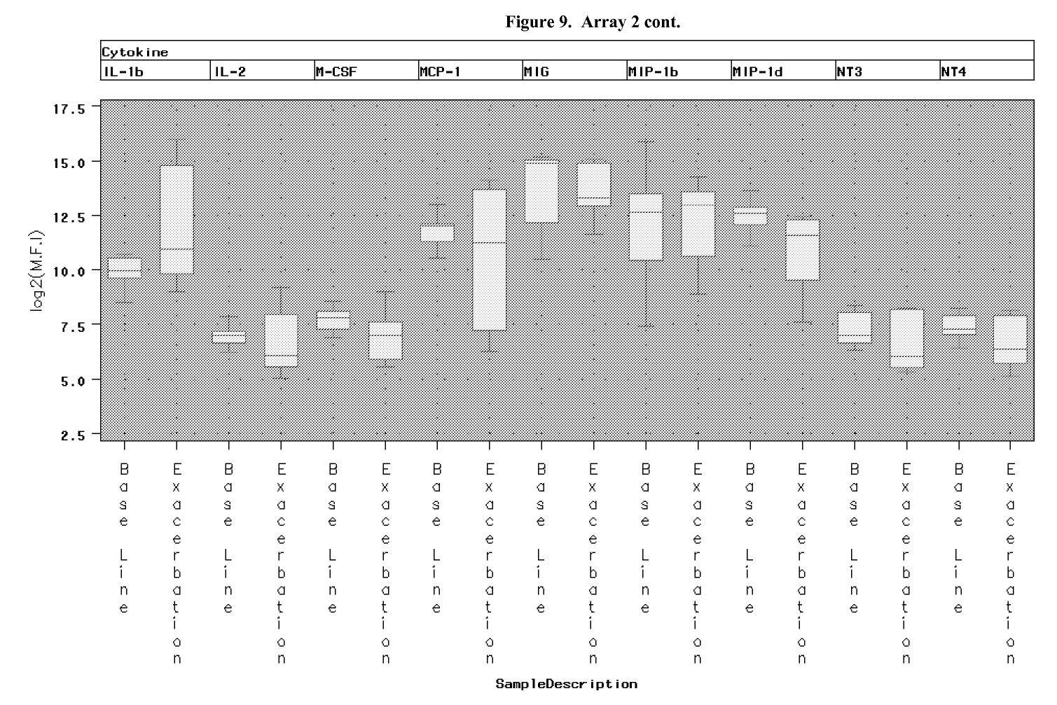

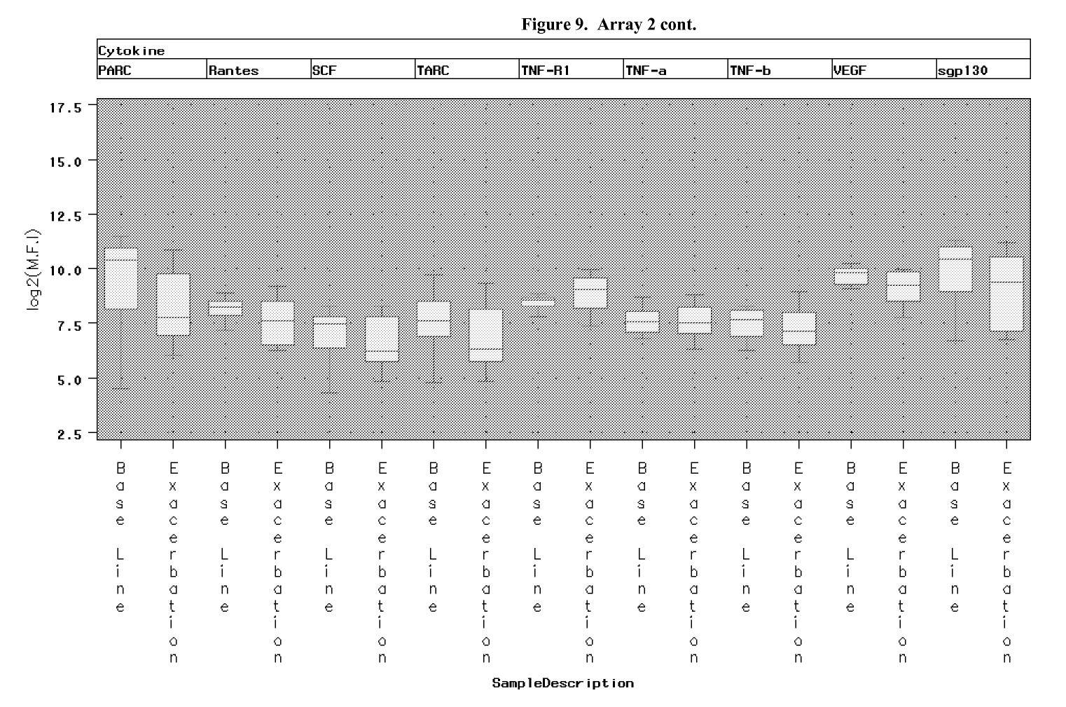

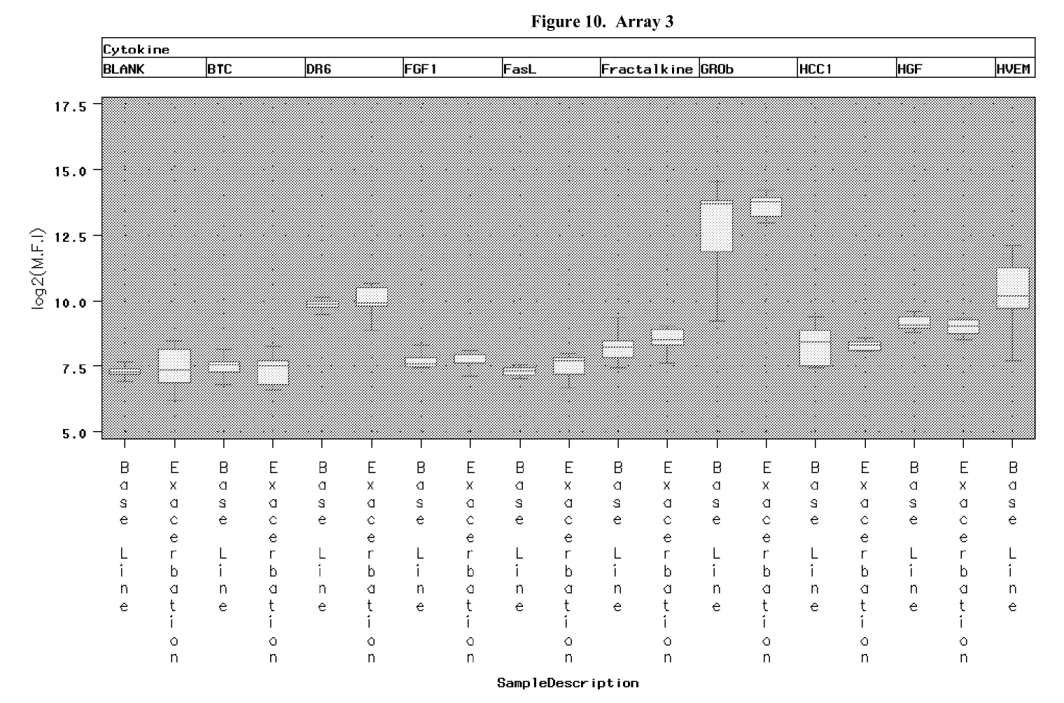

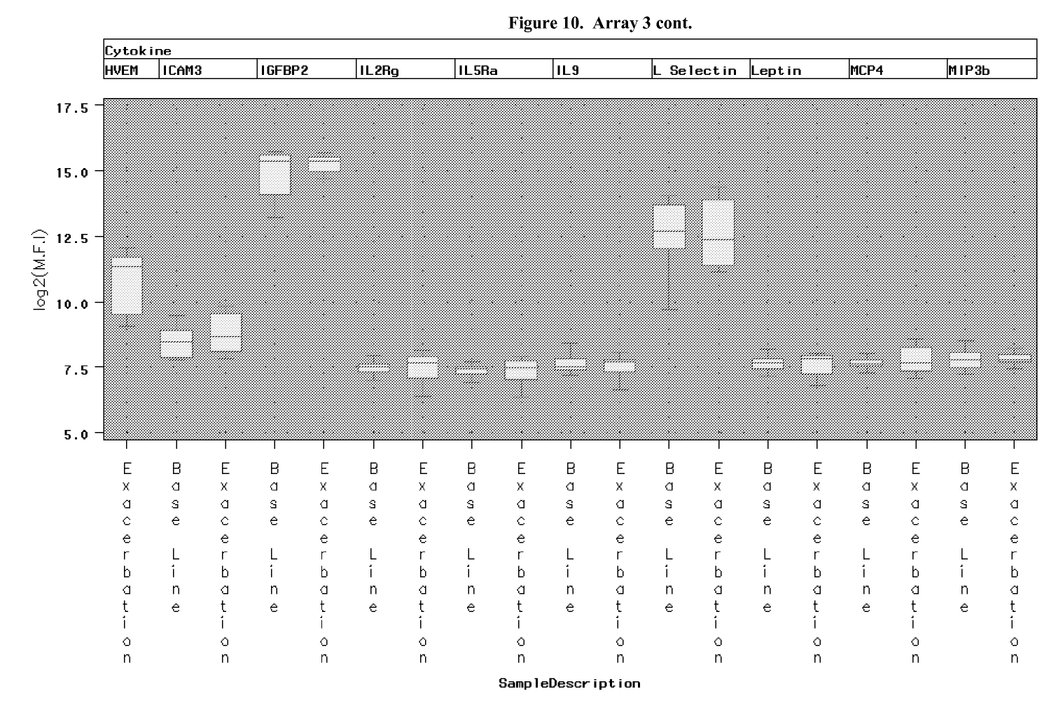

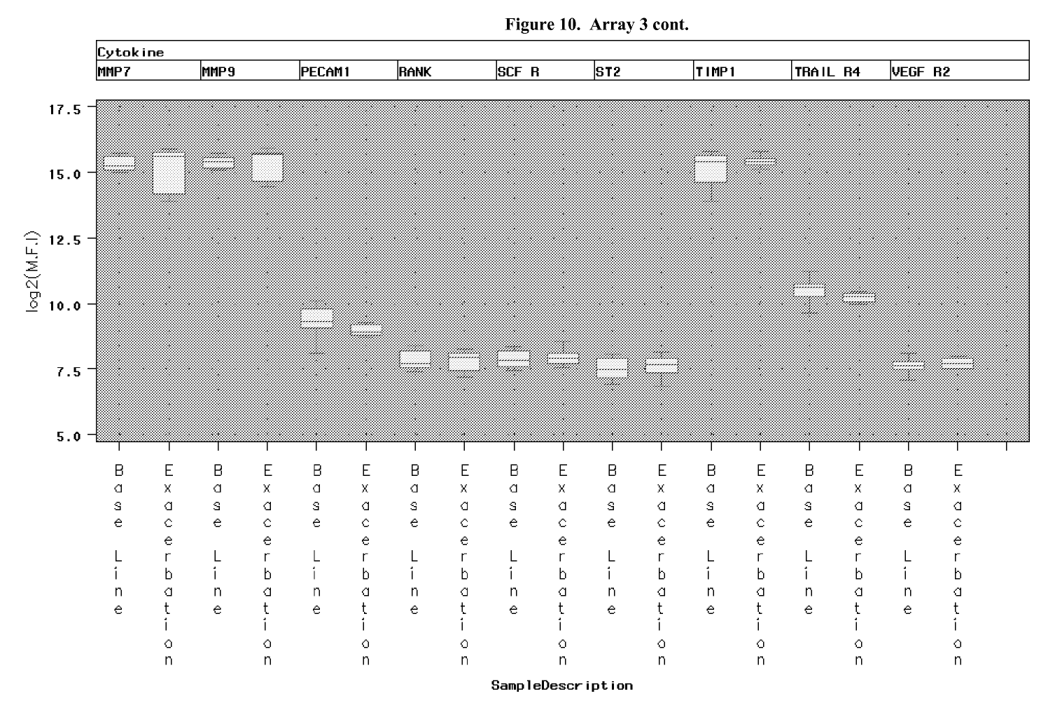

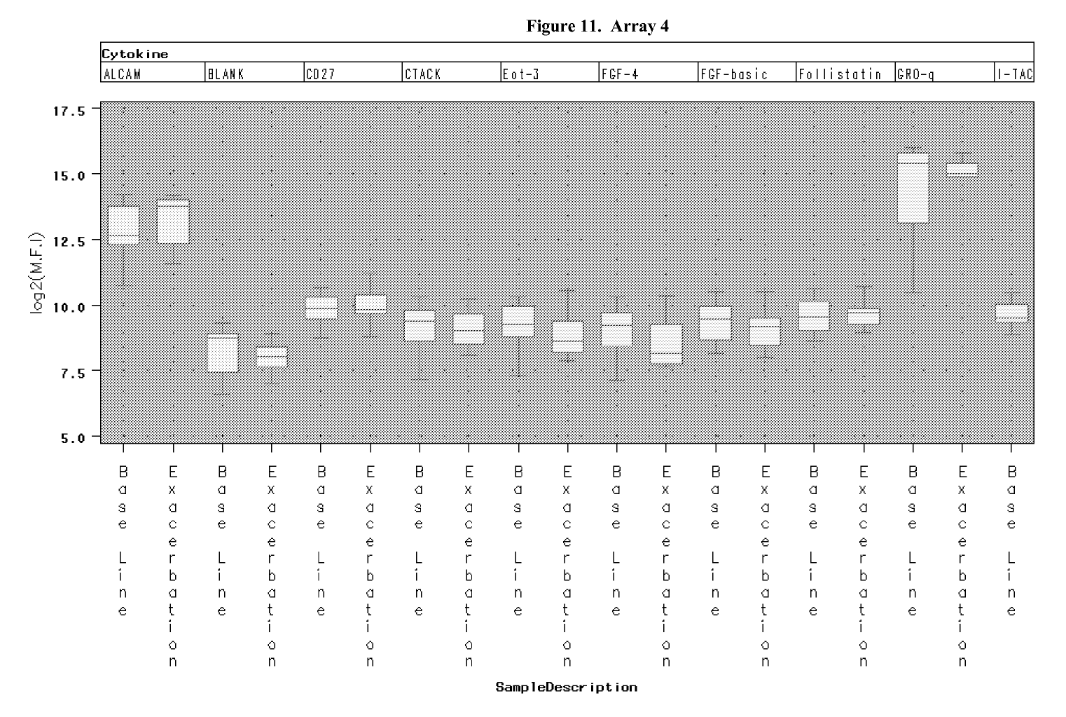

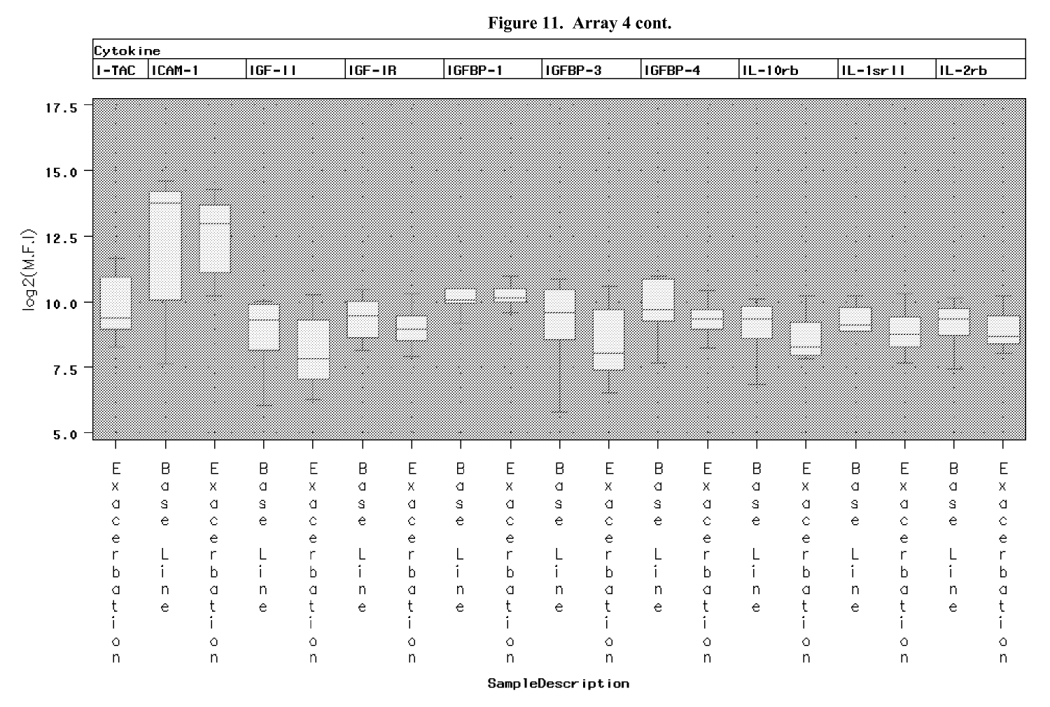

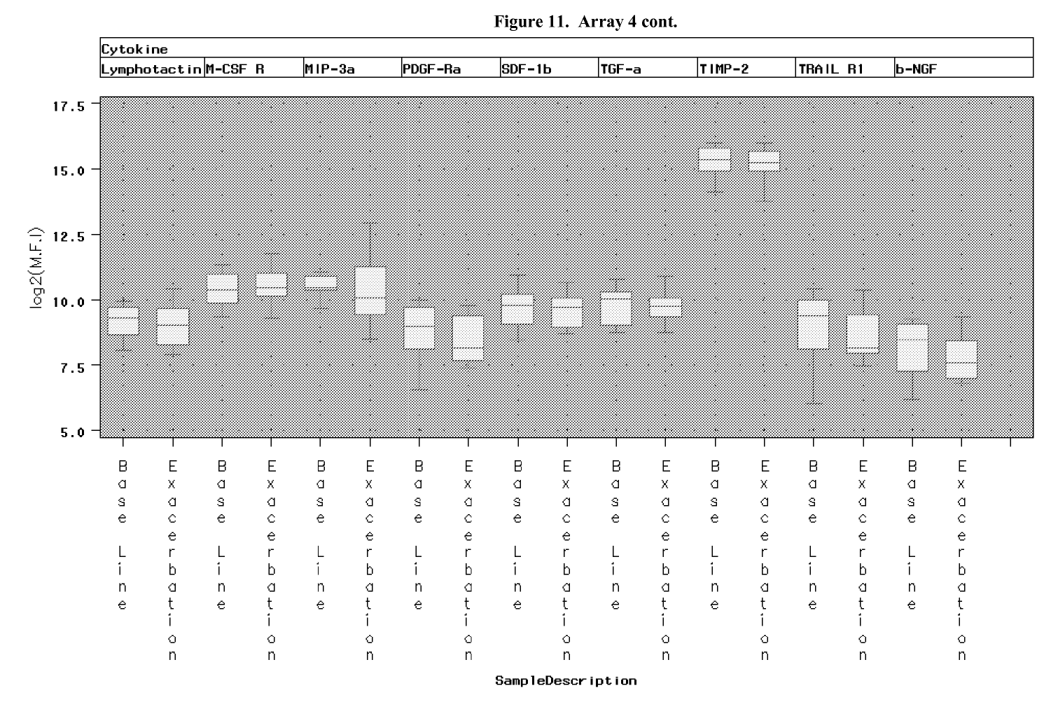

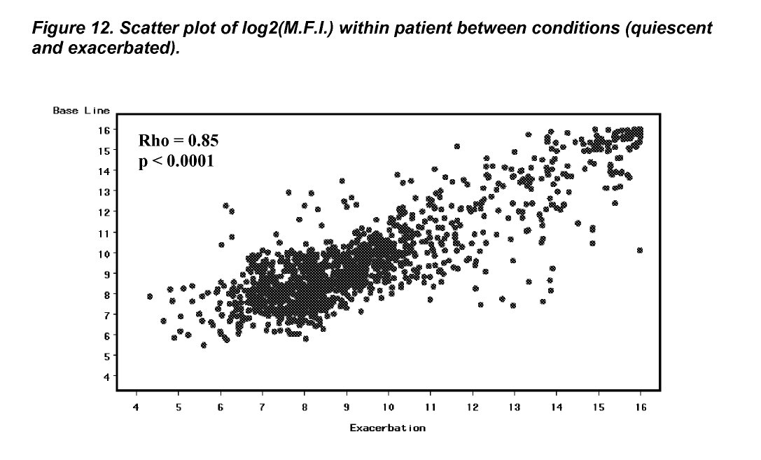

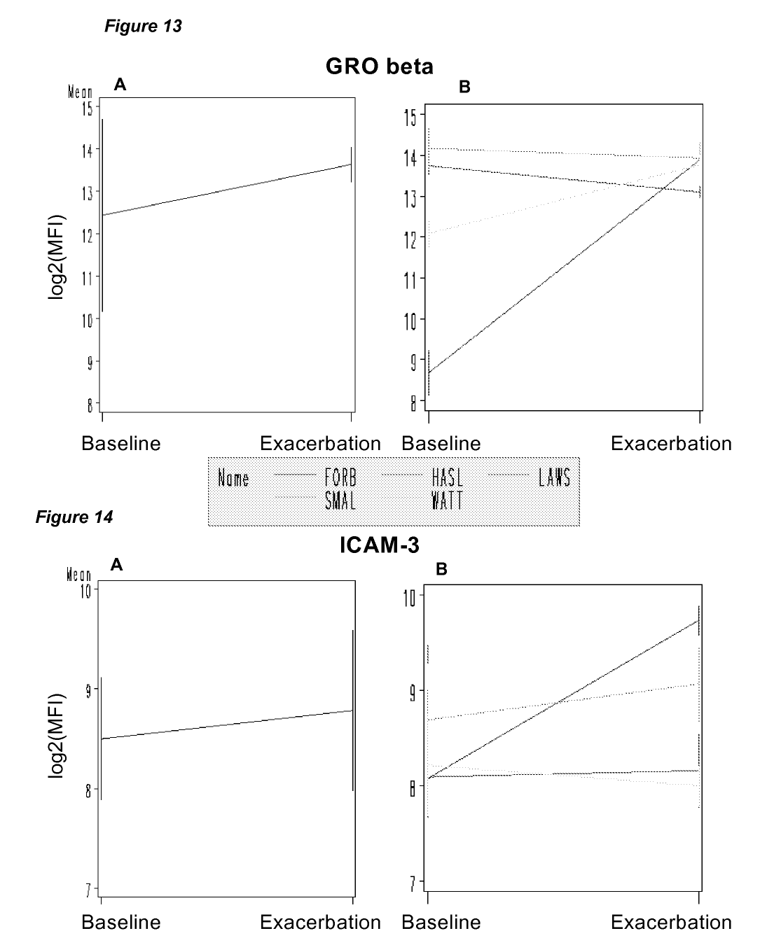

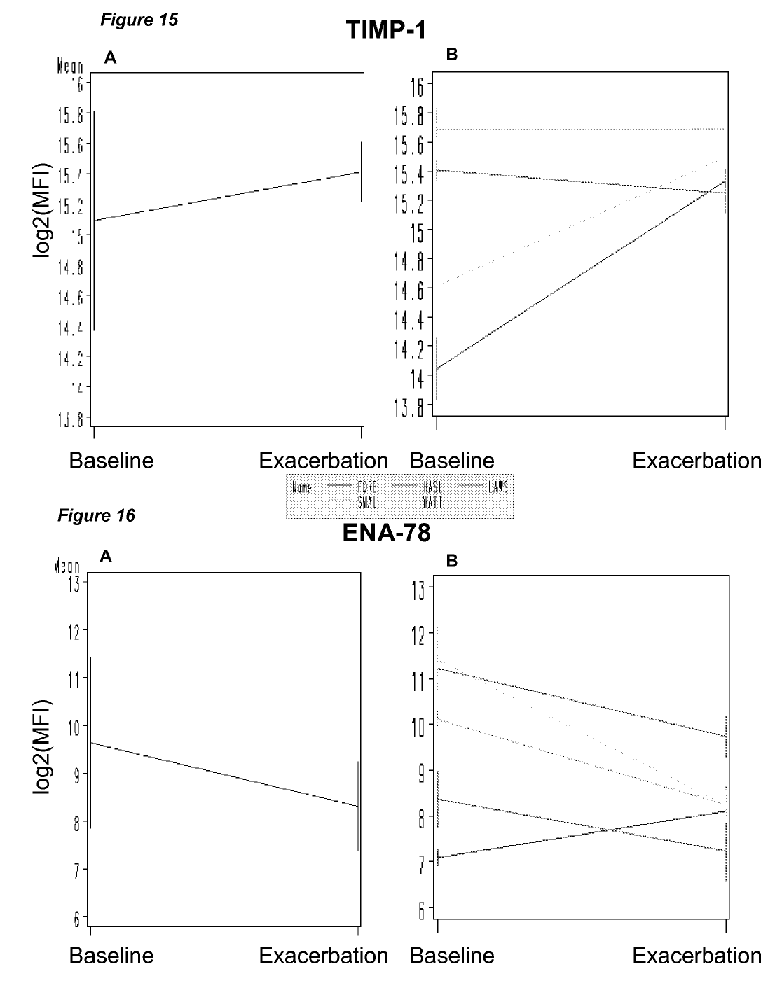

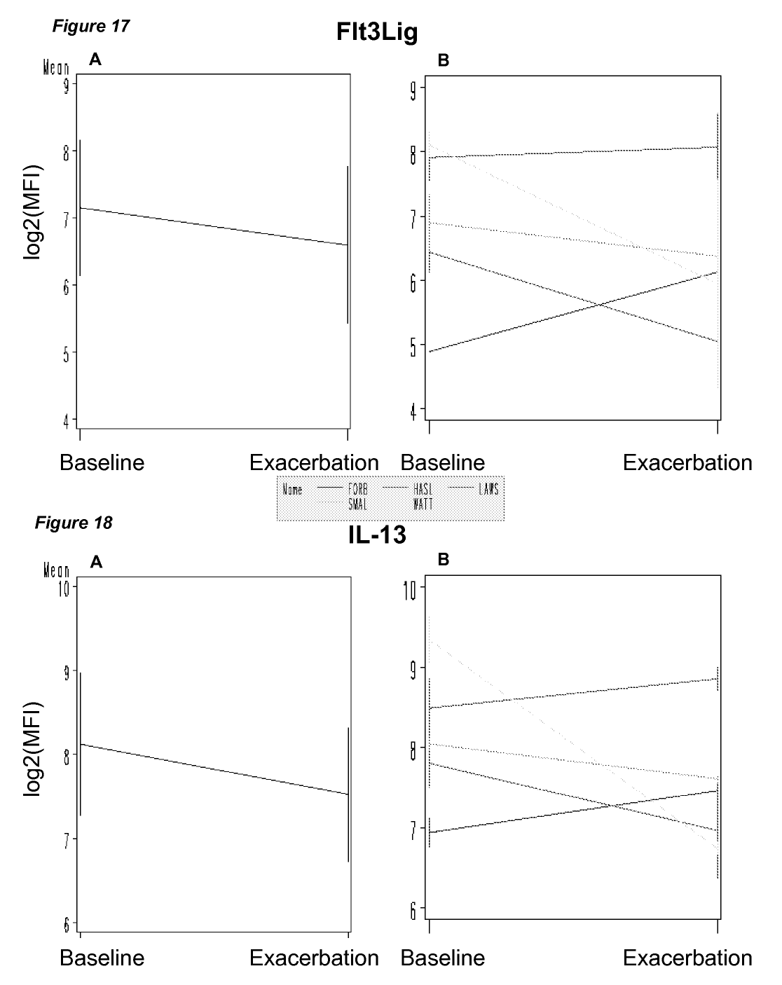

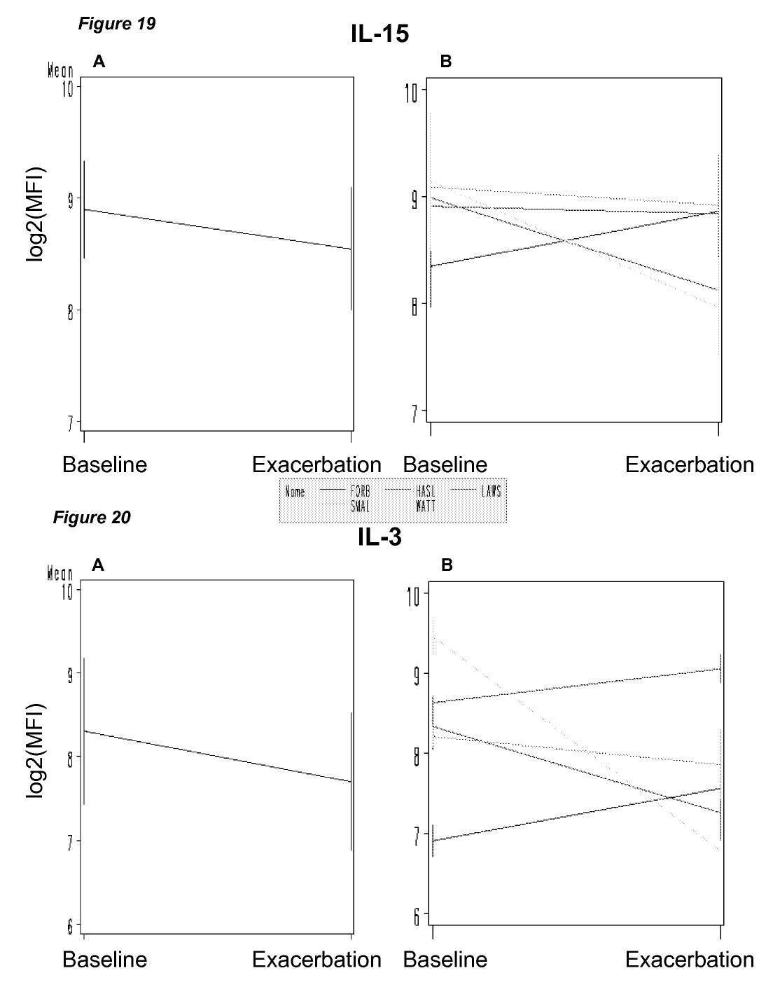

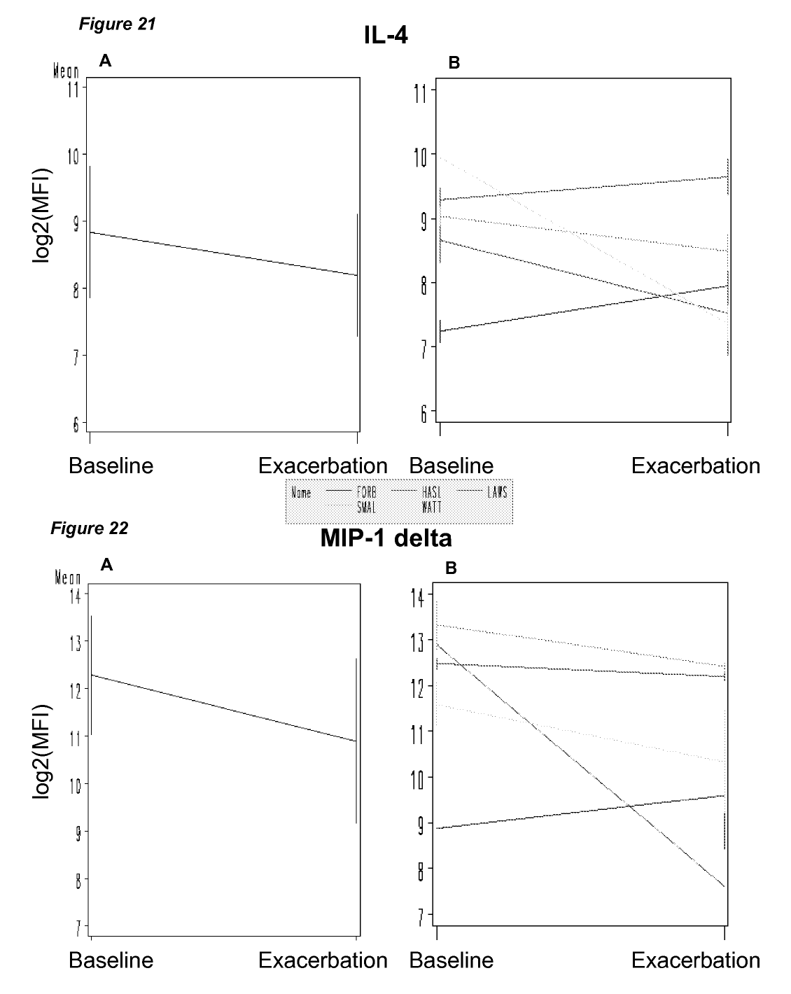

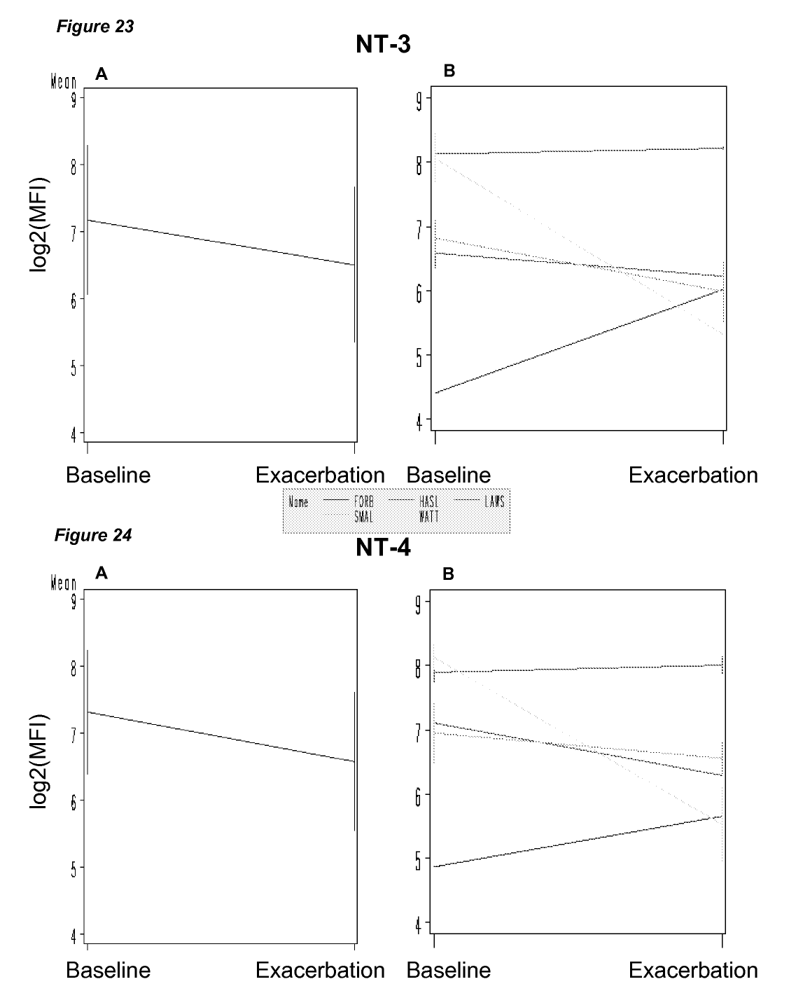

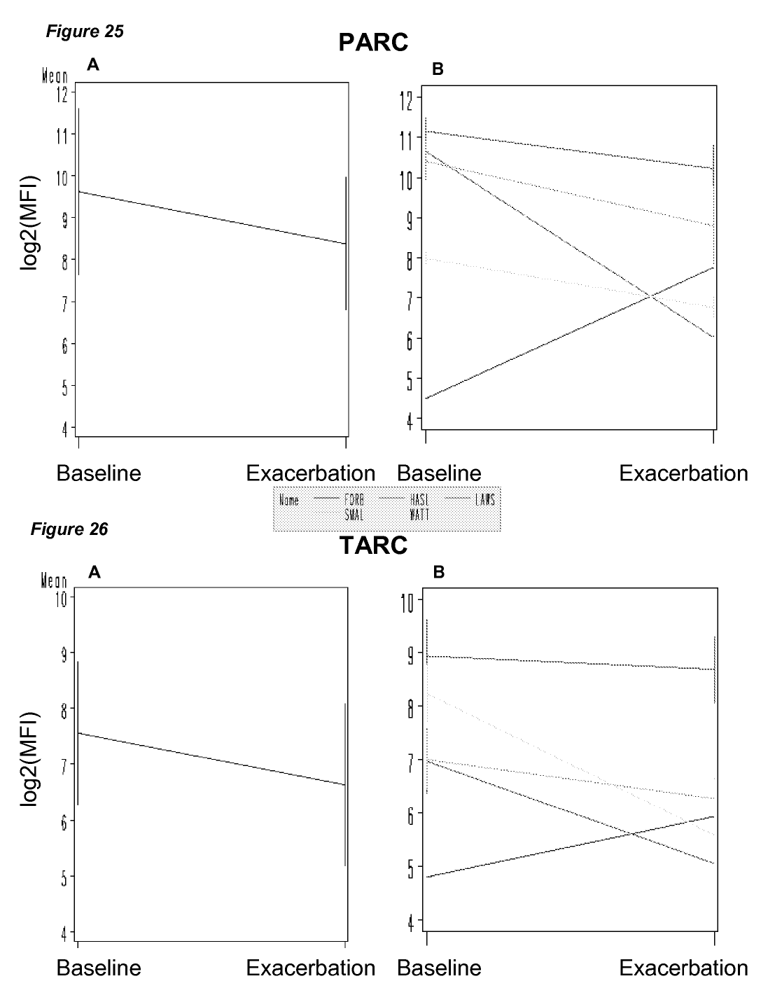

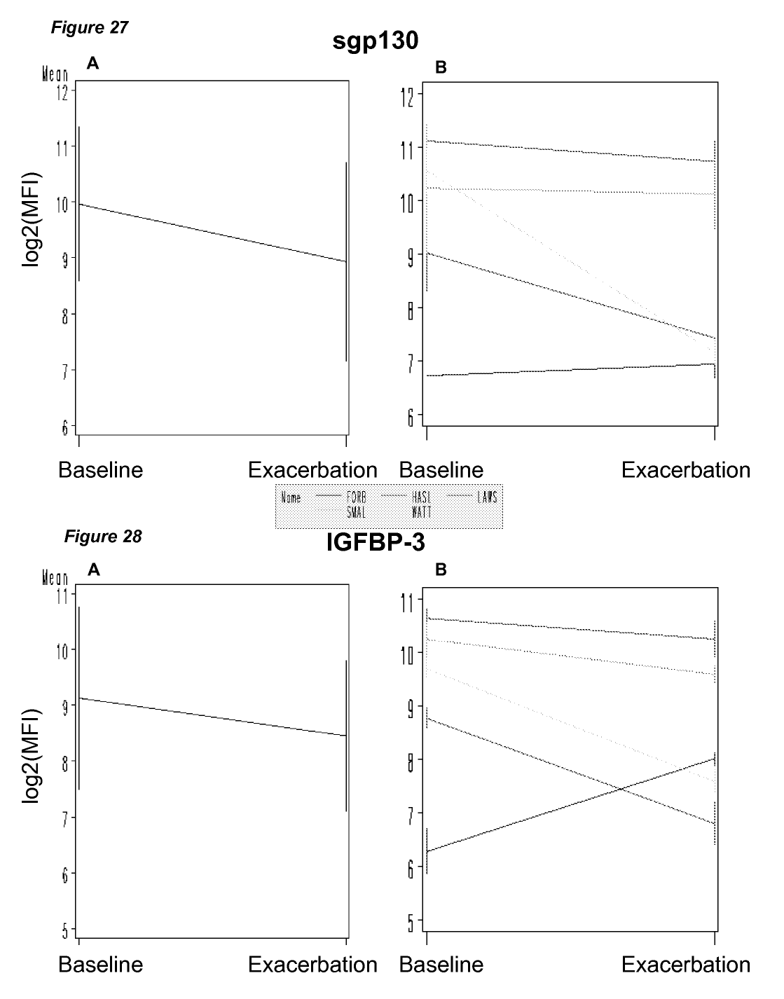

The results can be summarized as follows:
1. 95% of GSK sample replicates passed QC, indicating that sputum is a compatible sample type for MSI protein microarrays. Precision analysis revealed the average CV between slides was between 14% and 35%.
2. Sixty-two of 107 analytes exhibited levels >1000 MFI (mean fluorescence intensity) in the quiescent sputum samples, indicating that these analytes can be detected in processed sputum samples (see Table 2).
3. Correlation analysis showed that analyte intensities within each patient were highly correlated between baseline and exacerbation (coefficient of correlation >0.85), indicating that inter-individual variation is an important contributor to analyte values in sputum and underscoring the value of longitudinal assessment with this sample type (see FIG. 12).
4. Sixteen analytes (GRO-β, ICAM-3, TIMP-1, ENA-78, Flt3Lig, IL-13, IL-15, IL-3, IL-4, MIP-1δ, NT3, NT4, PARC, TARC, sgp130, IGFBP-3) were found to be modulated in exacerbated COPD samples compared to baseline samples (Table 3, FIGS. 13-28). Some of these analytes have been shown to be associated with COPD, while others may represent novel findings.

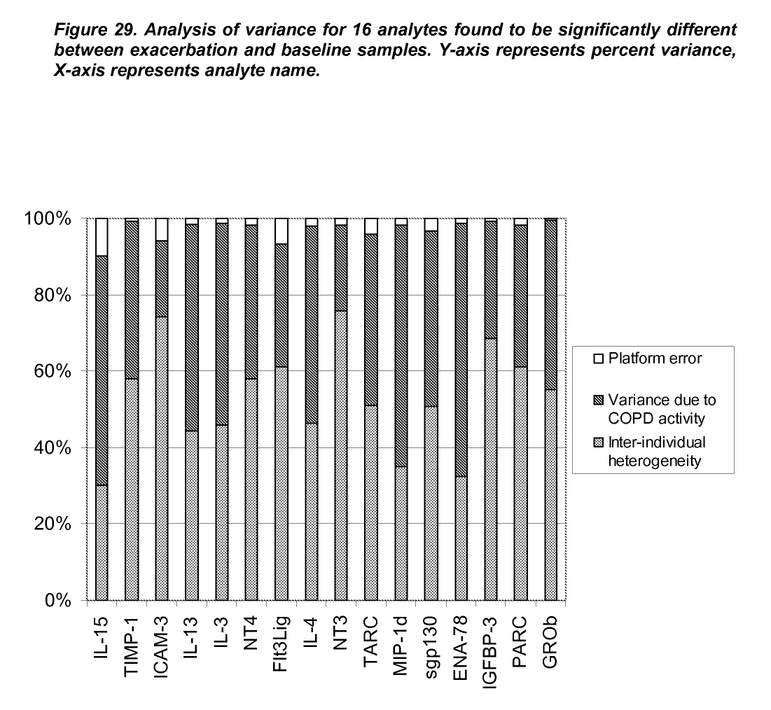

If each condition (quiescent and exacerbation) were assigned to different individuals, there would be no baseline available to adjust for variability observed between individual patients. Typically, this study type has lower precision requiring the condition effects to be much larger to be considered significant. It is possible to simulate such studies using repeated measure studies by treating the longitudinal component as a unique patient with a given condition (quiescent or exacerbated). Under these assumptions, variance component analysis was performed on the “cross-sectional study” (FIG. 29). A major source of variation was due to population differences. Variation due to condition (quiescent or exacerbated) was much lower and no significant differences were found.

Based on sputum compatibility and interesting findings revealed in this pilot study, a larger study that includes information about treatment effects would be worthwhile and may shed light on quantitative cytokine changes caused by disease activity, medication, and other variables.

Detection of Protein Analyte Expression

Molecular signatures associated with COPD, including those associated with COPD patients of a particular age or age range and/or gender, are determined by detecting expression in a test sample of at least one of the protein analytes disclosed below. Protein analytes can be detected in a test sample by any means known in the art. Any immunological detection method known in the art can be used. Solid phase immunoassays are particularly useful for this purpose. Methods that apply the power of nucleic acid signal amplification to the detection of non-nucleic acid analytes can be employed for detecting, determining, and quantitating specific protein analytes in samples. See U.S. Pat. No. 6,531,283, which is incorporated herein by reference.

Multiple proteins can be analyzed, for example, by sandwich immunoassays on microarrays to which primary antibodies specific to the various proteins have been immobilized. First, the protein analytes, if present in the sample, are captured on the cognate spots on the array by incubation of the sample with the microarray under conditions favoring specific antigen-antibody interactions. Second, a rolling circle amplification (RCA) primer is associated with the various protein analytes using a secondary antibody that is specific for the protein analyte being detected and which is conjugated to the RCA primer or a hapten. In direct immunoassays, the secondary antibody is conjugated directly to the RCA primer. In indirect immunoassays, the secondary antibody is conjugated to a hapten, such as biotin and then incubated with a detector antibody conjugate or streptavidin conjugated with the RCA primer. Rolling circle replication primed by the primers results in production of a large amount of DNA at the site in the array where the proteins are immobilized. The amplified DNA serves as a readily detectable signal for the proteins.

Different proteins in the array can be distinguished in several ways. For example, the location of the amplified DNA can indicate the protein involved, if different proteins are immobilized at pre-determined locations in the array. Alternatively, each different protein can be associated with a different rolling circle replication primer that in turn primes rolling circle replication of a different DNA circle. The result is distinctive amplified DNA for each different protein. The different amplified DNAs can be distinguished using any suitable sequence-based nucleic acid detection technique. Comparison of protein analytes found in two or more different samples can be performed using any means known in the art. For example, a first sample can be analyzed in one array and a second sample analyzed in a second array that is a replica of the first array. The intensity of a spot for each protein analyte at the first array can be compared with the intensity of the corresponding spot of the second array. The differences in the intensities of the spot between the first and second array determine if the concentration of the protein analyte is different in the two samples. If differences exist, they are recorded as elevated protein analyte or depressed protein analyte. Alternatively, the same protein analyte(s) from different samples can be associated with different primers which prime replication of different DNA circles to produce different amplified DNAs. In this manner, each of many protein analytes present in several samples can be quantitated.

Protein analytes can be tested directly or derivatives of the protein analytes can be tested. The derivatives can be forms of the protein analyte which occur in the body, or forms which are produced, either spontaneously or by design, during sample processing. Examples of derivatives include proteolytic degradation products, phosphorylated products, acetylated products, myristoylated products, transaminated products, protein complexed products, and complex dissociated products. All such derivatives are included within the term “protein analyte.”

Quantitation of Protein Analyte Expression

A variety of different solid phase substrates can be used to quantitate or determine the concentration of a protein analyte. The choice of substrate can be readily made by the routineer, based on convenience, cost, skill, or other considerations. Useful substrates include without limitation: beads, bottles, surfaces, substrates, fibers, wires, framed structures, tubes, filaments, plates, sheets, and wells. These substrates can be made from: polystyrene, polypropylene, polycarbonate, glass, plastic, metal, alloy, cellulose, cellulose derivatives, nylon, coated surfaces, acrylamide or its derivatives and polymers thereof, agarose, or latex, or combinations thereof. This list is illustrative rather than exhaustive.

Other methods of protein detection and measurement described in the art can be used as well. For example, a single antibody can be coupled to beads or to a well in a microwell plate, and quantitated by immunoassay. In this assay format, a single protein analyte can be detected in each assay. The assays can be repeated with antibodies to many protein analytes to arrive at essentially the same results as can be achieved using the methods of this invention. Bead assays can be multiplexed by employing a plurality of beads, each of which is uniquely labeled in some manner. For example each type of bead can contain a pre-selected amount of a fluorophore. Types of beads can be distinguished by determining the amount of fluorescence (and/or wavelength) emitted by a bead. Such fluorescently labeled beads are commercially available from Luminex Corporation (Austin, Tex.) and permit up to 100 protein analyte measurements simultaneously.

Protein analytes can alternatively be measured by enzyme-linked immunosorbent assay (ELISA), which permits a single protein measurement per microwell, and can be scaled up to 384 or more measurements per plate. Non-immunological assays can also be used. Enzyme activity-based assays can achieve a high degree of sensitivity and can be used. Specific binding protein assays can be used where a protein is a member of a specific binding pair that has a high binding affinity (low dissociation constant). The other member of the specific binding pair may be a protein or a non-protein, such as a nucleic acid sequence which is specifically bound by a protein.

Some protein analytes may be informative of COPD or of patient condition when considered in isolation. However, more typically expression of a plurality of protein analytes will be tested and considered in determining a diagnosis or prognosis. Expression of 2, 3, 4, 5, 6, or 7 protein analytes may be considered. In some cases a larger number of protein analytes may be tested, but only a subset may be sufficient to provide a diagnosis or prognosis. It may be desirable in order to gain increased statistical power, to test an even larger number of protein analytes, such as at least 8, 9, 10, 12, 14, or 16 protein analytes. It may also be desirable to utilize both one or more protein analytes which are elevated and one or more protein analytes which are depressed in the same assay.

The concentration of a protein analyte in a test sample and the concentration of the protein analyte in a control (reference) sample is different if, taking into account the accuracy and sensitivity of the particular detection method used, the concentrations are statistically different. Statistical differences can be determined using statistical methods well known in the art (e.g., Student's t-test). Determination of accuracy and sensitivity is well within the skill of those in the art.

Test and Control Samples

Samples for testing according to the invention can be derived from any readily available patient material. Typically this will be a body fluid sample, such as blood, serum, or plasma. Other body samples can be used as well, including synovial fluid, cerebrospinal fluid, urine, sputum, tears, saliva, stool, biopsy, and cheek smear. Body samples can be fractionated prior to testing to improve sensitivity and reduce background. Any fractionation procedure known in the art can be used, so long as the desired analyte remains in the fraction which is used as a test sample.

Control samples can be derived from a healthy individual or individuals or from an individual or individuals who are ill but who do not have COPD. These samples can be assayed individually or in pools. The data from individual controls can be pooled to provide a range of “normal” values. The data can be obtained at an earlier time. Thus controls need not be run in a side-by-side fashion with test samples. For some purposes, samples from a single individual taken at different times are compared to each other. In such cases there need not be evaluated, but may be, any control or normal sample. Control samples can also be synthetically produced, by mixing known quantities of particular analytes, either in an artificial or a natural body sample fluid.

Drug Screening Methods

The invention also provides methods of screening test substances to identify those which may be useful for treating COPD. For example, a test sample obtained from a patient diagnosed with COPD can be contacted with a test substance to form a contacted test sample. A molecular signature for the contacted test sample can be determined as described herein. This molecular signature can then be compared with the molecular signature of a test sample obtained from the same or a different patient diagnosed with COPD but which was not contacted with the test substance. A test substance preferably affects expression of one or more protein analytes. More preferably, a test substance either decreases or increases levels of a protein analyte by a statistically significant amount (p≦0.05) relative to the expression of the protein analyte in the absence of the test substance. The test substance preferably changes expression of the protein analyte so that it more closely resembles the expression of the protein analyte in a control molecular signature. That is, if expression of a protein analyte is decreased in the molecular signature of a COPD patient relative to expression of the protein analyte in a control molecular signature, then the test substance preferably elevates the expression of the protein analyte in the contacted test sample. Similarly, if expression of a protein analyte is elevated in the molecular signature of an COPD patient relative to expression of the protein analyte in a control molecular signature, then the test substance preferably decreases the expression of the protein analyte in the contacted test sample.

Test Substances

Test substances can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test substances can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test substances can be screened for the ability to affect protein analyte expression or expression of a polynucleotide encoding the protein analyte using high throughput screening. Using high throughput screening, many discrete compounds can be tested in parallel so that large numbers of test substances can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released from the beads. Active compounds can be visualized as dark pigment areas because, as the compounds diffuse locally into the gel matrix, the active compounds cause the cells to change colors.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLE 1

### Microarray Manufacture

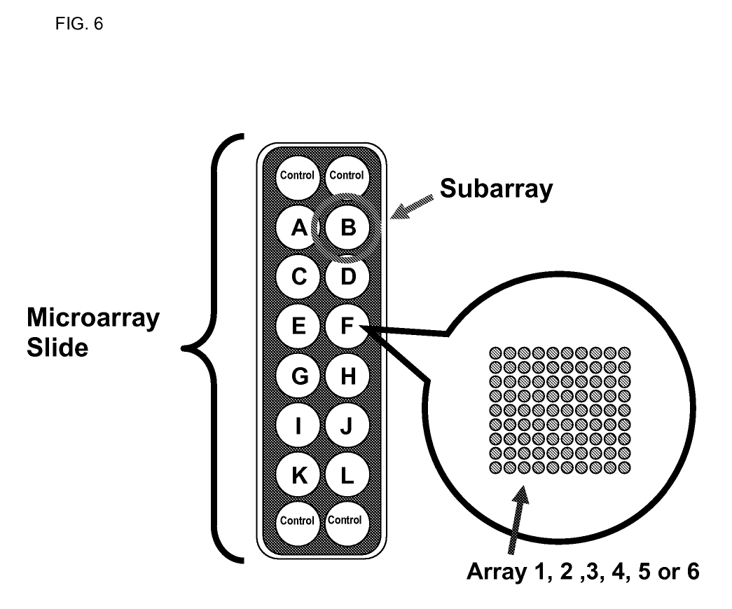

Glass slides were cleaned and derivatized with 3-cyanopropyltriethoxysilane. The slides were equipped with a Teflon mask that divided the slide into sixteen 0.65 cm diameter wells or circular analysis sites called subarrays (FIG. 6). Printing was accomplished with a Perkin-Elmer SpotArray Enterprise non-contact arrayer equipped with piezoelectric tips, which dispense a droplet (˜350 pL) for each microarray spot. Antibodies were applied at a concentration of 0.5 mg/mL at defined positions. Each chip was printed with sixteen copies of one type of array, either array 1, 2, 3, 4, 5 or 6. A set of antibodies as indicated in Table 12 below was printed with quadruplicate spots in each subarray. An exemplary microarray slide is depicted in FIG. 6.

After printing, chips were inspected using light microscopy. If the percentage of missing spots observed was greater than 5%, then the batch failed and the slides were discarded immediately. For all print runs described herein, 100% of the antibody features and >95% of the biotin calibrators were printed.

Microarray chips were validated in concert with a set of qualified reagents in two ways. First, mixtures of 1-3 different cytokines were prepared so as to provide a high intensity signal and applied to 14 wells of a chip (with each well being treated with a different mixture up to the total complement of detector antibodies). Two arrays were used as blank controls. The chips were developed and scanned, and the resulting signals were compared to the positional map of the particular array. Second, a titration quality control for all protein analytes of a specified array using known sample matrices was performed. Normal human serum and heparinized plasma were assayed either neat or spiked with purified recombinant cytokines representing all protein analytes in the array. Spiked mixtures were then titrated down the subarrays of a slide from 9,000 pg/mL to 37 pg/mL of spiked cytokine concentrations along with two subarrays for each un-spiked control sample. The data was quantified, and for every protein analyte in the array a titration curve was generated to examine feature intensity behavior as a function of concentration. Taken together, this data was used to confirm the activity of array features and reagent sets.

### EXAMPLE 2

### RCA Immunoassay

Prior to assay, the slides were removed from storage at room temperature in sealed containers and opened in a humidity controlled chamber (45-55%). Slides were blocked with Seablock (Pierce Chemical Co.), diluted 1:1 with PBS for 1 h at 37° C. in a humidified chamber. Following removal of the blocking solution, they were washed twice with 1×PBS/0.5% Brij 35 prior to application of sample. Four controls were included on each sample slide with feature concentrations corresponding to four anchor points on the full titration curve. The test samples were assayed on the remaining 12 subarrays.

Twenty μL of the treated sample were applied to each subarray. The basics of performing immunoassays with RCA signal amplification are described in Nat. Biotechol. (2002) 20:359-65). Slides were scanned using a LS200 scanner (TECAN). The fluorescence intensity of microarray spots was analyzed for each feature and sample, and the resulting mean intensity values were determined. Dose-response curves for selected cytokines were examined, ensuring that feature intensity was above background and exhibited increasing intensity with increasing protein analyte concentration.

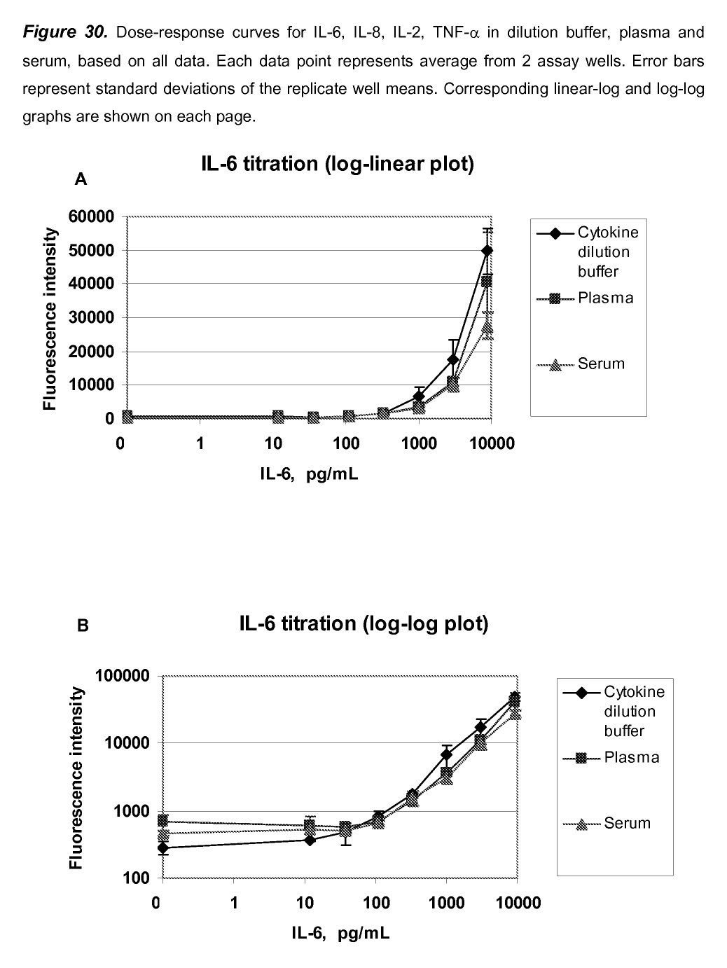

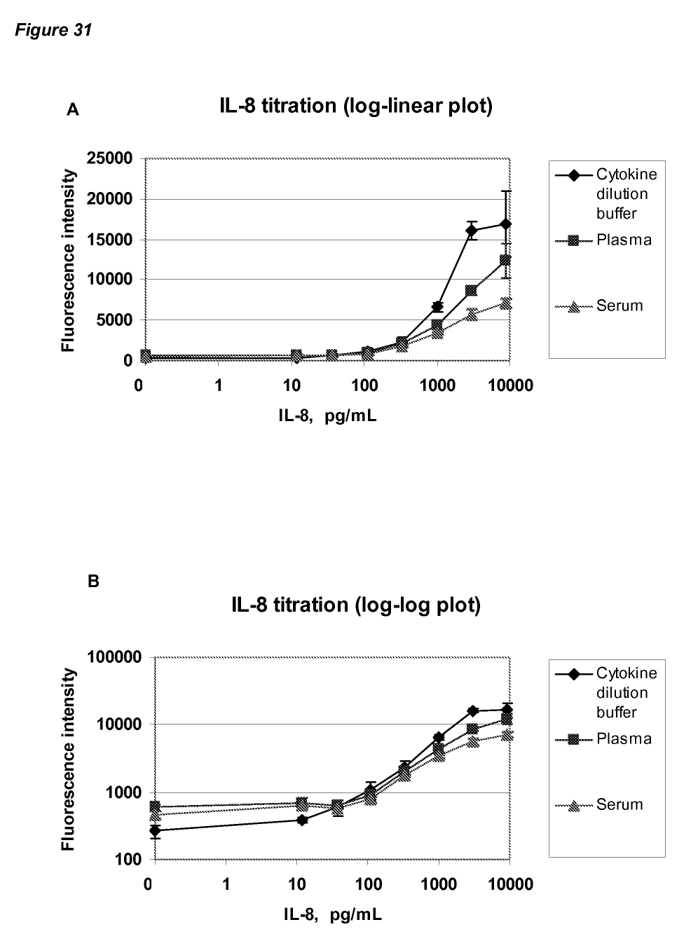

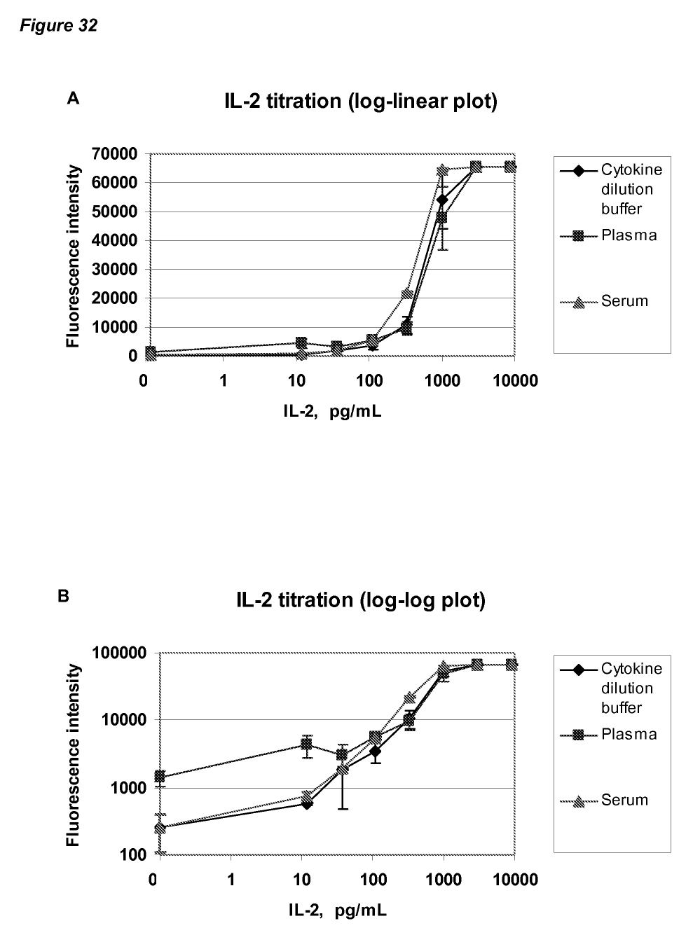

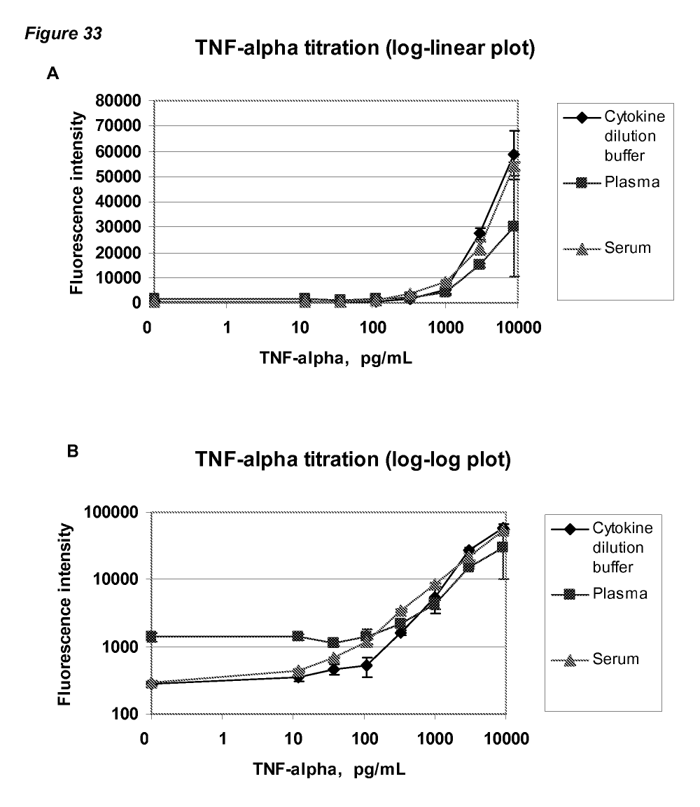

Control titrations of 4 exemplary analytes to demonstrate the working range are depicted in FIGS. 30-33.

### EXAMPLE 3

### Sample Grouping Statistics

Levels of 142 proteins were determined in 95 serum samples from COPD (47) and healthy controls (48). Each group was further divided into non-smokers (40 healthy controls, 39 COPD) and smokers (8 healthy controls, 8 COPD) (Table 1).

TABLE 1
Grouping statistics.
Patient Statistics

COPD exacerbations per year

| Smoking | CTRL | 0 | >0 <= 2 | >2 | Total |
|---|---|---|---|---|---|
| No | 40 | 12 | 12 | 15 | 79 |
| Yes | 8 | 4 | 4 | 0 | 16 |
| Total | 48 | 16 | 16 | 15 | 95 |

Data Quality

More than 94 percent of the samples passed MSI quality control (Table 2), exceeding the 85% minimum acceptable pass rate, indicative of successful completion of data generation according to MSI SOP.

TABLE 2
Sample pass rate by array.

| Array | Pass Rate |
|---|---|
| 1 | 95.8% |
| 2 | 99.3% |
| 3 | 96.8% |
| 4 | 94.4% |
| 5 | 98.9% |

Precision Assessment

Slide-to-slide imprecision was reduced using regression-based normalization. Slide-to-slide-variability (CV) was 24%, 24%, 27%, 22%, 23% on average, for Array 1, 2, 3, 4 and 5, respectively (Table 3). These values are consistent with standard platform performance.

TABLE 3
Slide-to-slide variability on a linear scale (CV in %).

| Array | <CV> | N | Std Dev |
|---|---|---|---|
| 1 | 24.36 | 2575 | 14.67 |
| 2 | 24.15 | 2565 | 14.66 |
| 3 | 27.23 | 2652 | 18.24 |
| 4 | 21.91 | 3606 | 14.21 |
| 5 | 22.61 | 2610 | 13.06 |

Biomarker Discovery

Thirty-eight of the 142 analytes tested showed no change and were excluded from statistical analysis. Analysis of variance (ANOVA) was used to test the significance of the different hypotheses using the GLM procedure of SAS. Reported effect size measures the difference in mean between two groups, normalized by within group standard deviation, and is independent of the sample size:
Effect Size=(Mean_Group1_Mean_Group2)/Std_Group1_Group2

Effect size has a direct association with the predictive ability of a particular variable. Table 4 shows conversions of effect sizes (column 1) to probability (column 2). The example presented in Table 4 is intended to demonstrate the relationship between effect size and predictive ability. For example, with an effect size of 0.3 observed between the two groups, the probability of correctly identifying the groups is 0.56. With an effect size of 1, the probability increases to 0.69.

TABLE 4
Effect size as the measure of predictive ability.

| Effect | Probability that grouping could be correctly |
|---|---|
| Size | assigned based on protein expression |
| 0 | 0.5 |
| 0.1 | 0.52 |
| 0.2 | 0.54 |
| 0.3 | 0.56 |
| 0.4 | 0.58 |
| 0.5 | 0.6 |
| 0.6 | 0.62 |
| 0.7 | 0.64 |
| 0.8 | 0.66 |
| 0.9 | 0.67 |
| 1 | 0.69 |
| 1.2 | 0.73 |
| 1.4 | 0.76 |
| 1.6 | 0.79 |
| 1.8 | 0.82 |
| 2 | 0.84 |
| 2.5 | 0.89 |
| 3 | 0.93 |

In our experience an effect size equal to or greater than of 0.6 provides a good balance between predictive power and number of analytes.

### EXAMPLE 4

### Differentiation of COPD Subjects from Both Smoking and Non-Smoking Controls Using Serum Analyte Profiles and Identify Biomarkers for COPD

Analytes showing a significant difference in expression between COPD (n=47) and non-smoking healthy individuals (n=40) are shown in Table 5.

TABLE 5
Analytes showing a significant difference between COPD and non-smoking
controls. Data was sorted in decreasing order of effect size.

| | COPD | | | Non Smoking Controls | | | Difference | | Effect | |
|---|---|---|---|---|---|---|---|---|---|---|
| Analyte | Mean | Std Dev | N | Mean | Std Dev | N | Mean | Std Dev | Size | p-value |
| MMP9 | 12.48 | 0.58 | 47 | 11.84 | 0.58 | 40 | 0.64 | 0.58 | 1.11 | 2E−06 |
| MMP-10 | 10.58 | 0.74 | 47 | 9.9 | 0.51 | 40 | 0.68 | 0.65 | 1.05 | 5E−06 |
| Eot2 | 12.42 | 1 | 47 | 11.37 | 1.07 | 40 | 1.06 | 1.03 | 1.03 | 7E−06 |
| TARC | 8.04 | 1.03 | 47 | 7.22 | 0.6 | 40 | 0.82 | 0.86 | 0.96 | 2.5E−05 |
| MMP7 | 10.2 | 0.78 | 47 | 9.56 | 0.57 | 40 | 0.64 | 0.69 | 0.92 | 4.8E−05 |
| Neut Elast | 12.2 | 0.37 | 47 | 11.87 | 0.37 | 40 | 0.33 | 0.37 | 0.88 | 9.2E−05 |
| IL-8 | 7.97 | 0.5 | 47 | 7.48 | 0.62 | 40 | 0.49 | 0.56 | 0.87 | 0.00011 |
| MIF | 9.82 | 0.64 | 47 | 9.38 | 0.33 | 40 | 0.44 | 0.52 | 0.85 | 0.00017 |
| IL-10rb | 9.67 | 0.46 | 47 | 9.29 | 0.43 | 40 | 0.37 | 0.45 | 0.84 | 0.0002 |
| Eot | 8.06 | 0.34 | 47 | 7.72 | 0.5 | 40 | 0.35 | 0.42 | 0.83 | 0.00023 |
| MMP-8 | 13.42 | 1.18 | 47 | 12.51 | 1 | 40 | 0.91 | 1.1 | 0.83 | 0.00023 |
| BDNF | 8.88 | 1.68 | 47 | 7.72 | 1.03 | 40 | 1.16 | 1.42 | 0.82 | 0.00028 |
| TIMP1 | 11.92 | 0.8 | 47 | 11.23 | 0.89 | 39 | 0.68 | 0.84 | 0.81 | 0.00031 |
| AR | 7.1 | 0.41 | 47 | 6.8 | 0.33 | 40 | 0.3 | 0.37 | 0.79 | 0.00044 |
| FGF-4 | 8.66 | 0.49 | 47 | 8.31 | 0.36 | 40 | 0.34 | 0.44 | 0.79 | 0.00043 |
| IGFBP-4 | 12.73 | 0.34 | 47 | 12.47 | 0.32 | 40 | 0.26 | 0.33 | 0.78 | 0.00049 |
| TNF-R1 | 9.2 | 0.95 | 47 | 8.44 | 1.07 | 40 | 0.77 | 1.01 | 0.76 | 0.00064 |
| BLC | 7.91 | 0.79 | 47 | 7.4 | 0.56 | 40 | 0.51 | 0.7 | 0.73 | 0.00109 |
| CTACK | 12.02 | 0.64 | 47 | 11.58 | 0.6 | 40 | 0.44 | 0.62 | 0.72 | 0.0013 |
| HCC4 | 13.21 | 0.4 | 47 | 12.92 | 0.43 | 40 | 0.3 | 0.41 | 0.72 | 0.00125 |
| IL-12p40 | 7.24 | 0.41 | 47 | 6.93 | 0.45 | 40 | 0.31 | 0.43 | 0.72 | 0.00125 |
| MCP-1 | 9.61 | 0.77 | 47 | 9.1 | 0.61 | 40 | 0.51 | 0.7 | 0.72 | 0.00117 |
| VEGF | 7.25 | 0.53 | 47 | 6.9 | 0.44 | 40 | 0.35 | 0.49 | 0.72 | 0.00127 |
| MPIF-1 | 9.49 | 0.63 | 47 | 9.03 | 0.71 | 40 | 0.47 | 0.67 | 0.69 | 0.00179 |
| HCC1 | 10.41 | 0.54 | 47 | 10.09 | 0.42 | 40 | 0.32 | 0.49 | 0.66 | 0.00301 |
| EGF | 7.54 | 0.67 | 47 | 7.12 | 0.65 | 40 | 0.42 | 0.66 | 0.64 | 0.00377 |
| MIP-1b | 7.55 | 0.64 | 47 | 7.16 | 0.57 | 40 | 0.39 | 0.61 | 0.64 | 0.00364 |
| Prolactin | 10.14 | 0.6 | 47 | 9.82 | 0.37 | 40 | 0.31 | 0.51 | 0.62 | 0.00515 |
| IL-2sRa | 12.32 | 0.83 | 47 | 11.84 | 0.74 | 40 | 0.48 | 0.79 | 0.61 | 0.00557 |
| ProteinC | 13.1 | 0.25 | 47 | 12.92 | 0.37 | 40 | 0.19 | 0.31 | 0.6 | 0.00651 |
| LT bR | 10.11 | 0.62 | 47 | 9.8 | 0.39 | 40 | 0.31 | 0.53 | 0.59 | 0.00785 |
| IGF-IR | 8.89 | 0.62 | 47 | 8.59 | 0.4 | 40 | 0.31 | 0.53 | 0.58 | 0.00897 |
| IL-17 | 8.55 | 0.31 | 47 | 8.37 | 0.29 | 40 | 0.18 | 0.3 | 0.58 | 0.00852 |
| MIG | 9.83 | 1.09 | 47 | 9.31 | 0.58 | 40 | 0.52 | 0.89 | 0.58 | 0.008 |
| IL-3 | 8.4 | 0.39 | 47 | 8.14 | 0.52 | 40 | 0.26 | 0.45 | 0.57 | 0.0097 |
| ICAM-1 | 13.88 | 0.43 | 47 | 13.62 | 0.5 | 40 | 0.26 | 0.46 | 0.56 | 0.0108 |
| GM-CSF | 8.38 | 0.4 | 47 | 8.12 | 0.55 | 40 | 0.26 | 0.48 | 0.55 | 0.01164 |
| IL-1srII | 9.1 | 0.54 | 47 | 8.82 | 0.49 | 40 | 0.28 | 0.52 | 0.55 | 0.01246 |
| ENA-78 | 10.27 | 1.01 | 47 | 9.68 | 1.15 | 40 | 0.59 | 1.08 | 0.54 | 0.01323 |
| MIP-1d | 11.25 | 0.68 | 47 | 10.9 | 0.57 | 40 | 0.35 | 0.64 | 0.54 | 0.01318 |
| PARC | 13.34 | 0.27 | 47 | 13.2 | 0.27 | 40 | 0.15 | 0.27 | 0.54 | 0.01456 |
| Rantes | 15.16 | 0.37 | 47 | 14.93 | 0.48 | 40 | 0.23 | 0.42 | 0.53 | 0.01497 |
| IGF-II | 13.52 | 0.39 | 47 | 13.3 | 0.43 | 40 | 0.21 | 0.41 | 0.52 | 0.018 |
| NT3 | 7.38 | 0.49 | 47 | 7.13 | 0.49 | 40 | 0.26 | 0.49 | 0.52 | 0.01782 |
| NT4 | 6.87 | 0.53 | 47 | 6.61 | 0.47 | 40 | 0.26 | 0.5 | 0.52 | 0.01849 |
| AgRP | 7.28 | 0.36 | 47 | 7.1 | 0.33 | 40 | 0.18 | 0.34 | 0.51 | 0.01912 |
| ALCAM | 12.88 | 0.34 | 47 | 12.73 | 0.27 | 40 | 0.15 | 0.31 | 0.5 | 0.02331 |
| IGFBP-3 | 11.86 | 0.74 | 47 | 11.49 | 0.77 | 40 | 0.37 | 0.75 | 0.49 | 0.02518 |
| IGFBP-6 | 13.25 | 0.3 | 47 | 13.11 | 0.28 | 40 | 0.14 | 0.29 | 0.49 | 0.02448 |
| CD40 | 7.07 | 0.51 | 47 | 6.86 | 0.38 | 40 | 0.21 | 0.45 | 0.46 | 0.03469 |
| Flt3Lig | 8.65 | 0.47 | 47 | 8.41 | 0.59 | 40 | 0.24 | 0.53 | 0.45 | 0.04026 |
| HCG | 7.55 | 0.51 | 47 | 7.33 | 0.45 | 40 | 0.22 | 0.48 | 0.45 | 0.03852 |
| VAP-1 | 13.25 | 0.68 | 47 | 12.98 | 0.5 | 40 | 0.27 | 0.61 | 0.45 | 0.04137 |
| Follistatin | 10.43 | 0.75 | 47 | 10.13 | 0.62 | 40 | 0.3 | 0.7 | 0.44 | 0.04602 |
| MIP3b | 7.31 | 1.24 | 47 | 6.86 | 0.61 | 40 | 0.44 | 1 | 0.44 | 0.04196 |
| PAI-II | 7.86 | 0.53 | 47 | 7.61 | 0.64 | 40 | 0.25 | 0.58 | 0.44 | 0.04441 |
| PECAM1 | 6.85 | 0.54 | 47 | 6.6 | 0.58 | 40 | 0.25 | 0.56 | 0.44 | 0.04357 |
| ProteinS | 10.73 | 0.43 | 47 | 10.53 | 0.45 | 40 | 0.19 | 0.44 | 0.44 | 0.04497 |
| TRAIL R4 | 6.98 | 0.38 | 47 | 6.81 | 0.4 | 40 | 0.17 | 0.39 | 0.44 | 0.04607 |
| PF4 | 15.13 | 0.22 | 47 | 15.23 | 0.25 | 40 | −0.1 | 0.23 | −0.45 | 0.04022 |

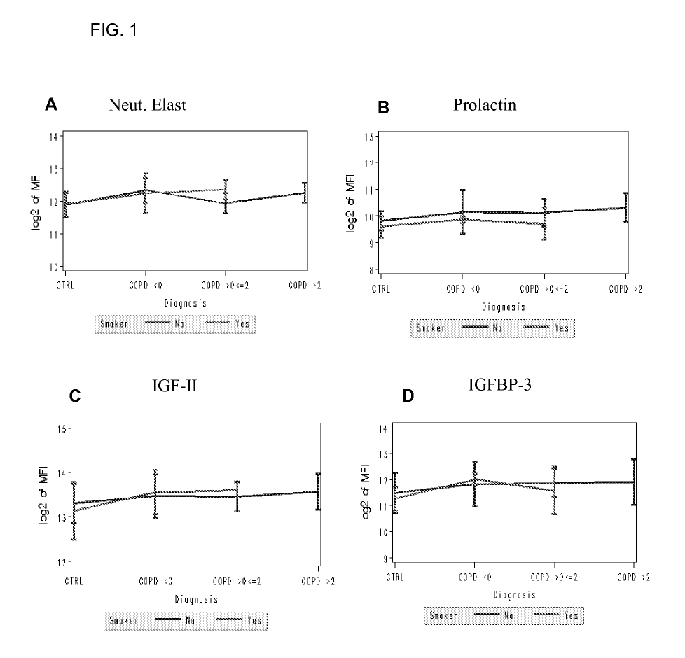

Similarly, analytes with a significant difference in expression observed between COPD (n=47) and smoking controls (n=8) are shown in Table 6 and FIG. 1.

TABLE 6
Analytes with significant differences observed between COPD and smoking
controls. Data sorted in decreasing order of effect size.

| | COPD | | | Smoking Controls | | | Difference | | Effect | |
|---|---|---|---|---|---|---|---|---|---|---|
| Analyte | Mean | Std Dev | N | Mean | Std Dev | N | Mean | Std Dev | Size | p-value |
| IGF-II | 13.52 | 0.39 | 47 | 13.13 | 0.65 | 8 | 0.38 | 0.44 | 0.88 | 0.0248 |
| IGFBP-3 | 11.86 | 0.74 | 47 | 11.27 | 0.44 | 8 | 0.58 | 0.7 | 0.83 | 0.034312 |
| Neut Elast | 12.2 | 0.37 | 47 | 11.91 | 0.37 | 8 | 0.29 | 0.37 | 0.78 | 0.046436 |
| Prolactin | 10.14 | 0.6 | 47 | 9.59 | 0.4 | 8 | 0.54 | 0.58 | 0.94 | 0.0177 |

All 4 analytes (IGF-II, IGFBP-3, neutrophil elastase and prolactin) were found in both Table 5 and Table 6 and might be specific to COPD due to independence of smoking.

### EXAMPLE 5

### Differentiation Between Frequent COPD Exacerbators (>2 Exacerbations Per Year), Infrequent Exacerbators (>0<=2 Exacerbations Per Year), and Non-Exacerbators (0 Exacerbations Per Year) Using a Serum Analyte Profile

These were planned comparisons and not analyzed as post hoc evaluations. Analysis could be confounded by the fact that there were no smokers in the frequent exacerbators group.

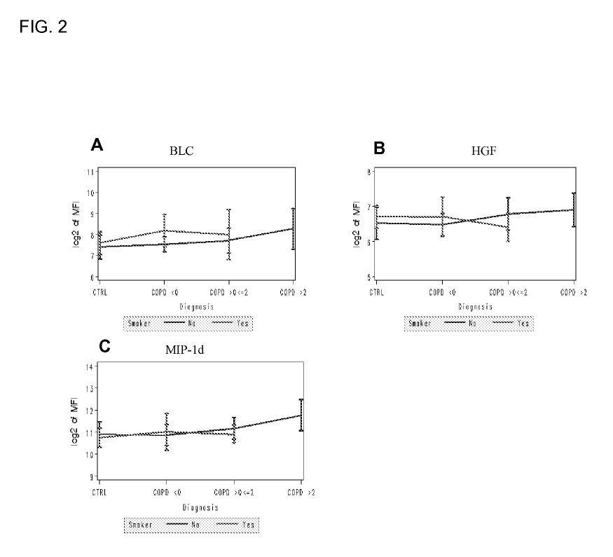

Table 7 and FIG. 2 show analytes with significant differences observed between COPD non-exacerbators (0 exacerbations) and frequent exacerbators (>2 exacerbations). All analytes were upregulated in exacerbators.

TABLE 7
Analytes with significant differences observed between
COPD non-exacerbators and frequent exacerbators.

| | Exacerbations per year | | | | | | Difference of | | | |
|---|---|---|---|---|---|---|---|---|---|---|
| | 0 | | | >2 | | | “0” and “>2” | | Effect | |
| Analyte | Mean | Std Dev | N | Mean | Std Dev | N | Mean | Std Dev | Size | p-value |
| BLC | 7.69 | 0.54 | 16 | 8.27 | 0.97 | 15 | −0.58 | 0.78 | −0.74 | 0.04829 |
| HGF | 6.53 | 0.38 | 16 | 6.9 | 0.47 | 15 | −0.36 | 0.43 | −0.84 | 0.02572 |
| MIP-1d | 10.9 | 0.56 | 16 | 11.78 | 0.71 | 15 | −0.87 | 0.63 | −1.38 | 0.00063 |

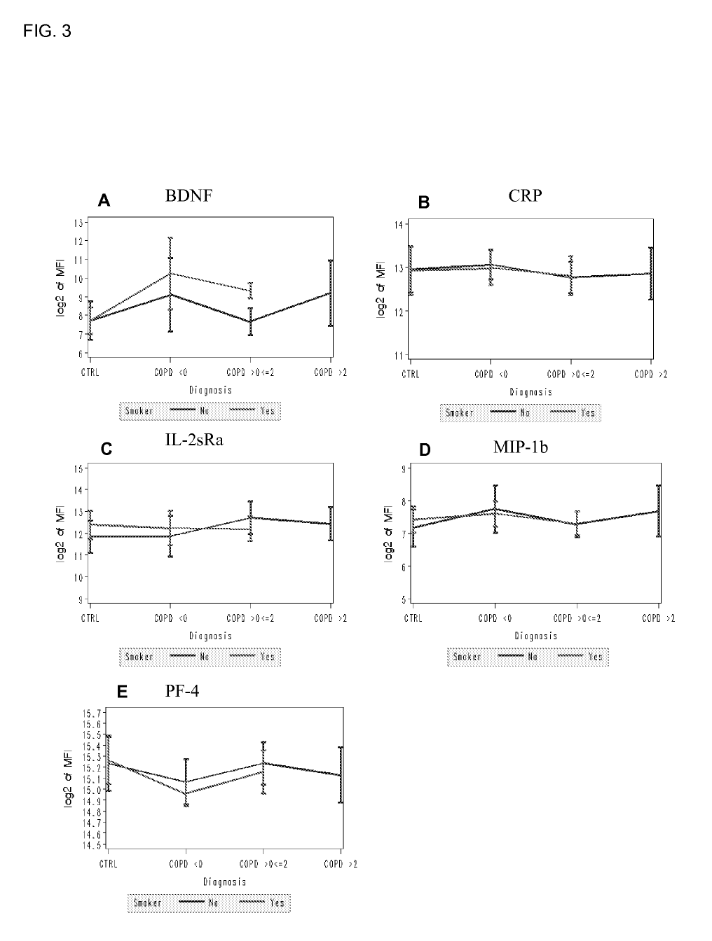

Table 8 and FIG. 3 show analytes with significant differences observed between COPD non-exacerbators (0 exacerbations) and infrequent exacerbators (>0<2 exacerbations).

TABLE 8
Analytes with significant differences observed between
COPD non-exacerbators and infrequent exacerbators.

| | Exacerbations per year | | | | | | Difference “0” | | | |
|---|---|---|---|---|---|---|---|---|---|---|
| | 0 | | | >0 <= 2 | | | and “>0 <= 2” | | Effect | |
| Analyte | Mean | Std Dev | N | Mean | Std Dev | N | Mean | Std Dev | Size | p-value |
| BDNF | 9.39 | 1.95 | 16 | 8.08 | 0.99 | 16 | 1.31 | 1.55 | 0.85 | 0.02273 |
| CRP | 13.04 | 0.35 | 16 | 12.78 | 0.36 | 16 | 0.27 | 0.35 | 0.75 | 0.0418 |
| IL-2sRa | 11.95 | 0.89 | 16 | 12.57 | 0.72 | 16 | −0.62 | 0.81 | −0.77 | 0.03837 |
| MIP-1b | 7.7 | 0.64 | 16 | 7.28 | 0.38 | 16 | 0.42 | 0.53 | 0.8 | 0.03182 |
| PF4 | 15.04 | 0.19 | 16 | 15.21 | 0.19 | 16 | −0.18 | 0.19 | −0.93 | 0.01364 |

With the exception of IL-2sRa and PF4, all analytes were upregulated in non-exacerbators. However, levels of PF4 were near saturation and results should be interpreted with caution.

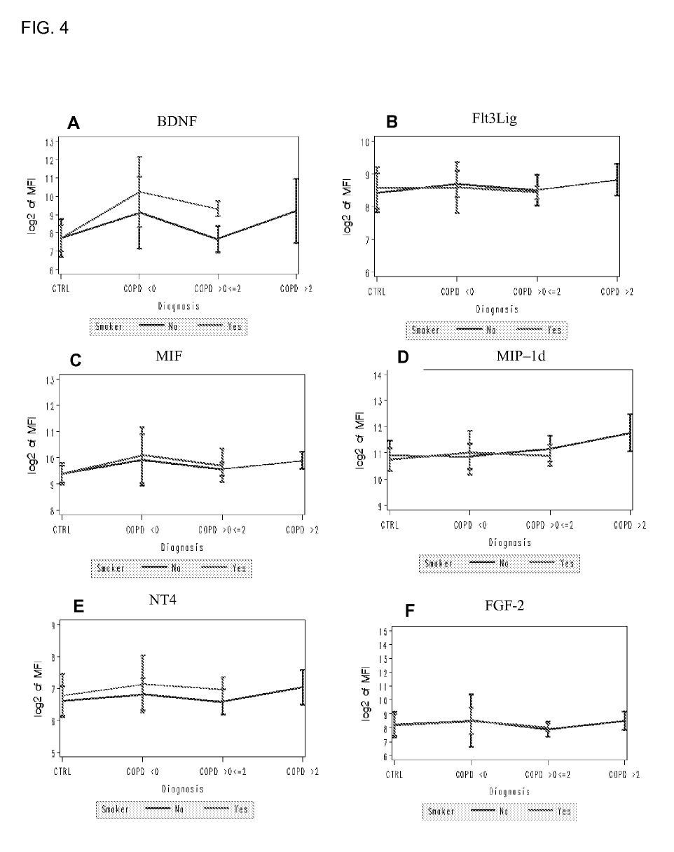

Table 9 and FIG. 4 show analytes with significant differences observed between COPD infrequent exacerbators (>0<2 exacerbations) and frequent exacerbators (>2 exacerbations).

TABLE 9
Analytes with significant differences observed between
COPD infrequent exacerbators and frequent exacerbators.

| | Exacerbations per year | | | | | | Difference “>0 <= | | | |
|---|---|---|---|---|---|---|---|---|---|---|
| | >0 <= 2 | | | >2 | | | 2” and “>2” | | Effect | |
| Analyte | Mean | Std Dev | N | Mean | Std Dev | N | Mean | Std Dev | Size | p-value |
| BDNF | 8.08 | 0.99 | 16 | 9.18 | 1.74 | 15 | −1.11 | 1.4 | −0.79 | 0.03666 |
| FGF-2 | 7.93 | 0.48 | 16 | 8.51 | 0.68 | 15 | −0.58 | 0.59 | −0.98 | 0.01059 |
| Flt3Lig | 8.48 | 0.42 | 16 | 8.82 | 0.48 | 15 | −0.34 | 0.45 | −0.76 | 0.0444 |
| MIF | 9.6 | 0.36 | 16 | 9.9 | 0.33 | 15 | −0.3 | 0.35 | −0.86 | 0.02313 |
| MIP-1d | 11.1 | 0.48 | 16 | 11.78 | 0.71 | 15 | −0.68 | 0.6 | −1.13 | 0.00383 |
| NT4 | 6.68 | 0.42 | 16 | 7.04 | 0.54 | 15 | −0.36 | 0.48 | −0.74 | 0.04733 |

### EXAMPLE 6

### Identification of Potential Changes in Analyte Profile that can be Attributed to Smoking

In this analysis all smokers were compared to all non-smokers. This type of analysis is confounded by clinical diagnosis, COPD or control. The results are shown in Table 10.

TABLE 10
Analysis of all smokers vs. all non-smokers.

| | | | | | | | Difference Non- | | | |
|---|---|---|---|---|---|---|---|---|---|---|
| | Non-Smoker | | | Smoker | | | Smoker and Smoker | | Effect | |
| Analyte | Mean | Std Dev | N | Mean | Std Dev | N | Mean | Std Dev | Size | p-value |
| CD141 | 9.2 | 0.58 | 79 | 8.88 | 0.44 | 16 | 0.32 | 0.56 | 0.58 | 0.03731 |
| ENA-78 | 9.91 | 1.09 | 79 | 10.94 | 1.35 | 16 | −1.02 | 1.14 | −0.9 | 0.00145 |
| ICAM-1 | 13.73 | 0.49 | 79 | 14.04 | 0.34 | 16 | −0.31 | 0.47 | −0.66 | 0.01731 |
| Leptin | 9.76 | 2.19 | 77 | 8.42 | 1.8 | 16 | 1.34 | 2.13 | 0.63 | 0.02467 |
| Prolactin | 10.01 | 0.54 | 79 | 9.69 | 0.4 | 16 | 0.32 | 0.52 | 0.62 | 0.02713 |
| TARC | 7.55 | 0.87 | 79 | 8.15 | 1.15 | 16 | −0.61 | 0.92 | −0.66 | 0.01826 |
| TIMP-2 | 14.57 | 0.25 | 79 | 14.73 | 0.21 | 16 | −0.16 | 0.24 | −0.66 | 0.01857 |

### EXAMPLE 7

### Non Confounded Analysis Using 2-Way ANOVA

As previously indicated, comparisons were confounded by either diagnosis or smoking. As an alternative, a 2-way ANOVA (Type III SS) was employed to investigate the smoking, COPD and smoking*COPD interaction.

An ANOVA model was created with 2 main effects and one interaction: Main effect: smoking (2 levels, smokers and non-smokers); Main effect: COPD (2 levels, controls and COPD); and Interaction: between smoking and COPD.

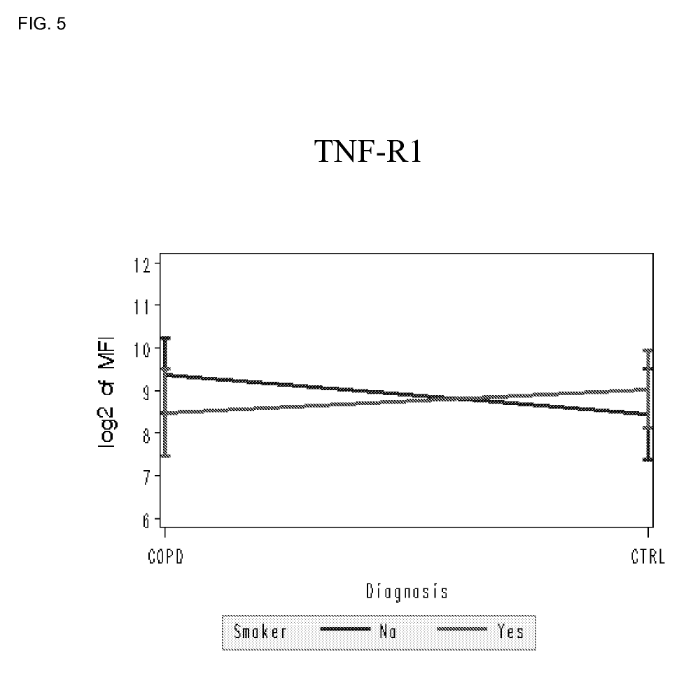

The results of the analysis are shown in Table 11. The “Main effects” interpretation is similar to a one-way ANOVA or t-test between two groups. Significant interaction indicates a difference in behavior between COPD and healthy controls for smokers and non-smokers. The TNF-R1 interaction plot is shown in FIG. 5. If the lines reflective of TNF-R1 values obtained with smokers and non-smokers for COPD and control are not parallel, this demonstrates that there is an interaction between COPD and smoking.

TABLE 11
Two-way ANOVA (Type III SS).

| Analyte | Effect | p-value |
|---|---|---|
| AgRP | COPD | 0.017534 |
| AR | COPD | 0.003016 |
| BDNF | COPD | 8.95E-05 |
| BLC | COPD | 0.012648 |
| Eot2 | COPD | 0.007806 |
| IGFBP-3 | COPD | 0.027088 |
| IGF-II | COPD | 0.007857 |
| IL-10rb | COPD | 0.010574 |
| IL-12p40 | COPD | 0.003009 |
| IL-17 | COPD | 0.001332 |
| IL-18 | COPD | 0.044281 |
| MCP-1 | COPD | 0.014996 |
| MIF | COPD | 0.001159 |
| MMP-10 | COPD | 0.010644 |
| MMP-8 | COPD | 0.000757 |
| MMP9 | COPD | 0.00059 |
| Neut Elast | COPD | 0.000963 |
| NT3 | COPD | 0.029722 |
| PF4 | COPD | 0.02277 |
| Prolactin | COPD | 0.03313 |
| TARC | COPD | 3.68E-05 |
| TIMP1 | COPD | 0.002852 |
| VEGF | COPD | 0.004115 |
| CD141 | Smoking | 0.037586 |
| ENA-78 | Smoking | 0.001394 |
| ICAM-1 | Smoking | 0.016017 |
| Leptin | Smoking | 0.022618 |
| MMP-10 | Smoking | 0.044882 |
| Prolactin | Smoking | 0.01893 |
| TARC | Smoking | 0.009814 |
| TIMP-2 | Smoking | 0.018618 |
| DR6 | Smoking*COPD | 0.040267 |
| HCC4 | Smoking*COPD | 0.031462 |
| HVEM | Smoking*COPD | 0.044902 |
| TNF-R1 | Smoking*COPD | 0.00697 |
| TRAIL R4 | Smoking*COPD | 0.021895 |

TABLE 12
Analytes on arrays 1-5.

| | |
|---|---|
| Analyte | Name |

Array 1 analytes

| | | |
|---|---|---|
| 1 | ANG | Angiogenin |
| 2 | BLC (BCA-1) | B-lymphocyte chemoattractant |
| 3 | EGF | Epidermal growth factor |
| 4 | ENA-78 | Epithelial cell-derived neutrophil-activating peptide |
| 5 | Eot | Eotaxin |
| 6 | Eot-2 | Eotaxin-2 |
| 7 | Fas | Fas (CD95) |
| 8 | FGF-7 | Fibroblast growth factor-7 |
| 9 | FGF-9 | Fibroblast growth factor-9 |
| 10 | GDNF | Glial cell line derived neurotrophic factor |
| 11 | GM-CSF | Granulocyte macrophage colony stimulating factor |
| 12 | IL-1ra | Interleukin 1 receptor antagonist |
| 13 | IL-2 sRα | Interleukin 2 soluble receptor alpha |
| 14 | IL-3 | Interleukin 3 |
| 15 | IL-4 | Interleukin 4 |
| 16 | IL-5 | Interleukin 5 |
| 17 | IL-6 | Interleukin 6 |
| 18 | IL-7 | Interleukin 7 |
| 19 | IL-8 | Interleukin 8 |
| 20 | IL-13 | Interleukin 13 |
| 21 | IL-15 | Interleukin 15 |
| 22 | MCP-2 | Monocyte chemotactic protein 2 |
| 23 | MCP-3 | Monocyte chemotactic protein 3 |
| 24 | MIP-1α | Macrophage inflammatory protein 1 alpha |
| 25 | MPIF | Myeloid progenitor inhibitory factor 1 |
| 26 | OSM | Oncostatin M |
| 27 | PIGF | Placental growth factor |

Array 2 analytes

| | | |
|---|---|---|
| 1 | AR | Amphiregulin |
| 2 | BDNF | Brain-derived neurotrophic factor |
| 3 | Flt-3 Lig | fms-like tyrosine kinase-3 ligand |
| 4 | GCP-2 | Granulocyte chemotactic protein 2 |
| 5 | HCC4 (NCC4) | Hemofiltrate CC chemokine 4 |
| 6 | I-309 | I-309 |
| 7 | IL-1α | Interleukin 1 alpha |
| 8 | IL-1β | Interleukin 1 beta |
| 9 | IL-2 | Interleukin 2 |
| 10 | IL-17 | Interleukin 17 |
| 11 | MCP-1 | Monocyte chemotactic protein 1 |
| 12 | M-CSF | Macrophage colony stimulating factor |
| 13 | MIG | Monokine induced by interferon gamma |
| 14 | MIP-1β | Macrophage inflammatory protein 1 beta |
| 15 | MIP-1δ | Macrophage inflammatory protein 1 delta |
| 16 | NT-3 | Neurotrophin 3 |
| 17 | NT-4 | Neurotrophin 4 |
| 18 | PARC | Pulmonary and activation-regulated chemokine |
| 19 | RANTES | Regulated upon activation, normal T expressed and presumably secreted |
| 20 | SCF | Stem cell factor |
| 21 | sgp130 | Soluble glycoprotein 130 |
| 22 | TARC | Thymus and activation regulated chemokine |
| 23 | TNF-RI | Tumor necrosis factor receptor I |
| 24 | TNF-α | Tumor necrosis factor alpha |
| 25 | TNF-β | Tumor necrosis factor beta |
| 26 | VEGF | Vascular endothelial growth factor |

Array 3 analytes

| | | |
|---|---|---|
| 1 | BTC | Betacellulin |
| 2 | DR6 | Death receptor 6 |
| 3 | Fas Lig | Fas ligand |
| 4 | FGF acid (FGF-1) | Fibroblast growth factor acidic |
| 5 | Fractalkine | Fractalkine |
| 6 | GRO-β | Growth related oncogene beta |
| 7 | HCC-1 | Hemofiltrate CC chemokine 1 |
| 8 | HGF | Hepatocyte growth factor |
| 9 | HVEM | Herpes virus entry mediator |
| 10 | ICAM-3 (CD50) | Intercellular adhesion molecule 3 |
| 11 | IGFBP-2 | Insulin-like growth factor binding protein 2 |
| 12 | L-2 Rγ | Interleukin 2 receptor gamma |
| 13 | IL-5 Rα (CD125) | Interleukin 5 receptor alpha |
| 14 | IL-9 | Interleukin 9 |
| 15 | Leptin/OB | Leptin |
| 16 | L-Selectin (CD62L) | Leukocyte selectin |
| 17 | MCP-4 | Monocyte chemotactic protein 4 |
| 18 | MIP-3β | Macrophage inflammatory protein 3 beta |
| 19 | MMP-7 (total) | Matrix metalloprotease 7 |
| 20 | MMP-9 | Matrix metalloprotease 9 |
| 21 | PECAM-1 (CD31) | Platelet endothelial cell adhesion molecule-1 |
| 22 | RANK | Receptor activator of NF-kappa-B |
| 23 | SCF R | Stem cell factor receptor |
| 24 | TIMP-1 | Tissue inhibitors of metalloproteases 1 |
| 25 | TRAIL R4 | TNF-related apoptosis-inducing ligand receptor 4 |
| 26 | VEGF-R2 (Flk-1/KDR) | Vascular endothelial growth factor receptor 2 |
| 27 | ST2 | Interleukin 1 receptor 4 |

Array 4 analytes

| | | |
|---|---|---|
| 1 | ALCAM | Activated leukocyte cell adhesion molecule |
| 2 | β-NGF | beta-nerve growth factor |
| 3 | CD27 | CD27 |
| 4 | CTACK | Cutaneous T-cell attracting chemokine |
| 5 | CD30 | CD30 |
| 6 | Eot-3 | Eotaxin-3 |
| 7 | FGF-2 | Fibroblast growth factor-2 (FGF-basic) |
| 8 | FGF-4 | Fibroblast growth factor-4 |
| 9 | Follistatin | Follistatin |
| 10 | GRO-γ | Growth related oncogene gamma |
| 11 | ICAM-1 | Intercellular adhesion molecule 1 |
| 12 | IFN-γ | Interferon gamma |
| 13 | IFN-ω | Interferon omega |
| 14 | IGF-1R | Insulin-like growth factor I receptor |
| 15 | IGFBP-1 | Insulin-like growth factor binding protein 1 |
| 16 | IGFBP-3 | Insulin-like growth factor binding protein 3 |
| 17 | IGFBP-4 | Insulin-like growth factor binding protein 4 |
| 18 | IGF-II | Insulin-like growth factor II |
| 19 | IL-1 sR1 | Interleukin 1 soluble receptor I |
| 20 | IL-1 sRII | Interleukin 1 soluble receptor II |
| 21 | IL-10 Rβ | Interleukin 10 receptor beta |
| 22 | IL-16 | Interleukin 16 |
| 23 | IL-2 Rβ | Interleukin 2 receptor beta |
| 24 | I-TAC | Interferon gamma-inducible T cell alpha chemoattractant |
| 25 | Lptn | Lymphotactin |
| 26 | LT βR | lymphotoxin-beta receptor |
| 27 | M-CSF R | Macrophage colony stimulating factor receptor |
| 28 | MIP-3α | Macrophage inflammatory protein 3 alpha |
| 29 | MMP-10 | Matrix metalloprotease 10 |
| 30 | PDGF Rα | Platelet-derived growth factor receptor alpha |
| 31 | PF4 | Stromal cell-derived factor beta |
| 32 | sVAP-1 | Soluble Vascular Adhesion Protein-1 |
| 33 | TGF-α | Transforming growth factor alpha |
| 34 | TIMP-2 | Tissue inhibitors of metalloproteases 2 |
| 35 | TRAIL R1 | TNF-related apoptosis-inducing ligand receptor 1 |
| 36 | VE-cadherin | Vascular Endothelial Cadherin |
| 37 | VEGF-D | Vascular endothelial growth factor-D |

Array 5 analytes

| | | |
|---|---|---|
| 1 | 4-1BB (CD137) | 4-1BB |
| 2 | ACE-2 | Angiotensin I converting enzyme-2 |
| 3 | AFP | Alpha fetoprotein |
| 4 | AgRP | Agouti-related protein |
| 5 | CD141 | Thrombomodulin/CD141 |
| 6 | CD40 | CD40 |
| 7 | CNTF Rα | Ciliary neurotrophic factor receptor alpha |
| 8 | CRP | C-reactive protein |
| 9 | D-Dimer | D-Dimer |
| 10 | E-Selectin | E-selectin |
| 11 | HCG | Human chorionic gonadotrophin |
| 12 | IGFBP-6 | Insulin-like Growth Factor Binding Protein 6 |
| 13 | IL-12 (p40) | Interleukin 12 p40 |
| 14 | IL-18 | Interleukin 18 |
| 15 | LIF Rα (gp190) | Leukemia inhibitory factor souble receptor alpha |
| 16 | MIF | Macrophage migration inhibitory factor |
| 17 | MMP-8 (total) | Matrix Metalloprotease-8 |
| 18 | NAP-2 | Neutrophil Activating Peptide 2 |
| 19 | Neutrophil elastase | Neutrophil elastase |
| 20 | PAI-II | Plasminogen activator inhibitor-II |
| 21 | Prolactin | Prolactin |
| 22 | Protein C | Human Protein C |
| 23 | Protein S | Human Protein S |
| 24 | P-Selectin | P-Selectin |
| 25 | TSH | Thyroid stimulating hormone |

### EXAMPLE 8

### Profile 107 Respiratory System Cytokine Levels in Sputum During an Exacerbation of COPD

The main objective of this project was to profile cytokine levels modulated in the respiratory system during symptomatic exacerbation of COPD. Testing sputum samples enables evaluation of localized cytokine modulation in the respiratory system. In the case of respiratory diseases such as COPD, localized cytokine levels in the respiratory system are likely to be representative of the pathophysiology of the disease and exacerbation of symptoms.

Testing was performed on 10 samples, an exacerbated COPD and a quiescent sample (baseline) from each of five patients. It was hypothesized that various cytokines would be modulated in these patients during exacerbation of the disease. Project goals were to (1) evaluate the compatibility of GSK's sputum samples with MSI's protein microarrays (Stage 1); and measure levels of 107 analytes (arrays 1-4) in processed sputum samples and provide the client with fluorescence intensity values (Stage 2).

Precision Analysis

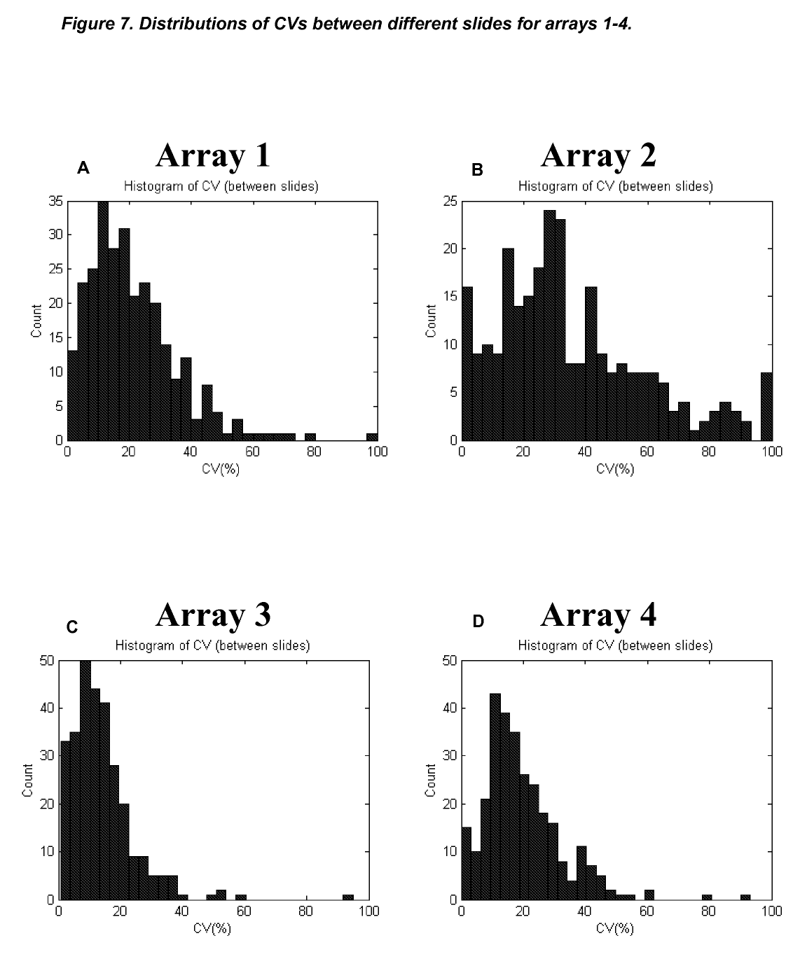

In order to assess the precision of the data obtained in this study, CVs between different slides were calculated on normalized data (Table 13). The average CV was 21%, 14%, and 20% between slides for arrays 1, 2, 3, and 4, respectively. The CVs observed for array 2 in this study were higher than the ˜20% CV typically observed across all arrays. The distributions of CVs between different slides for arrays 1-4 are shown in FIG. 7.

TABLE 13
Slide-to-slide variation of normalized fluorescence intensity units.

| Array # | <CV>¹ | STD (CV)² |
|---|---|---|
| 1 | 21% | 15% |
| 2 | 35% | 25% |
| 3 | 14% | 11% |
| 4 | 20% | 13% |

¹average CV
²Standard deviation of CV distribution

### EXAMPLE 9

### Analytes Detected in Quiescent Sputum Samples

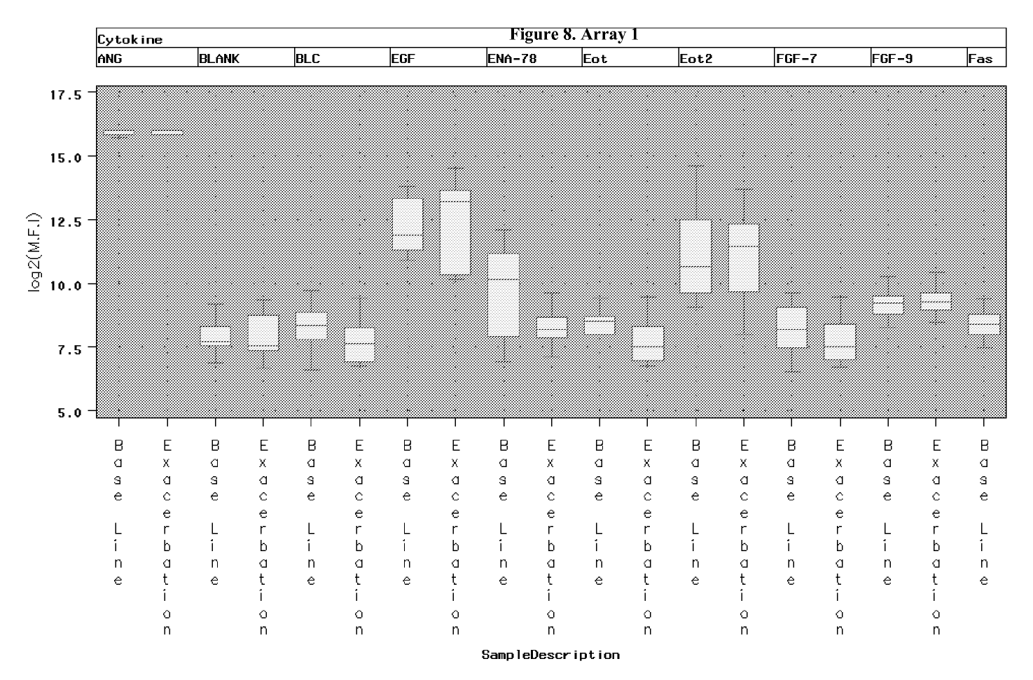

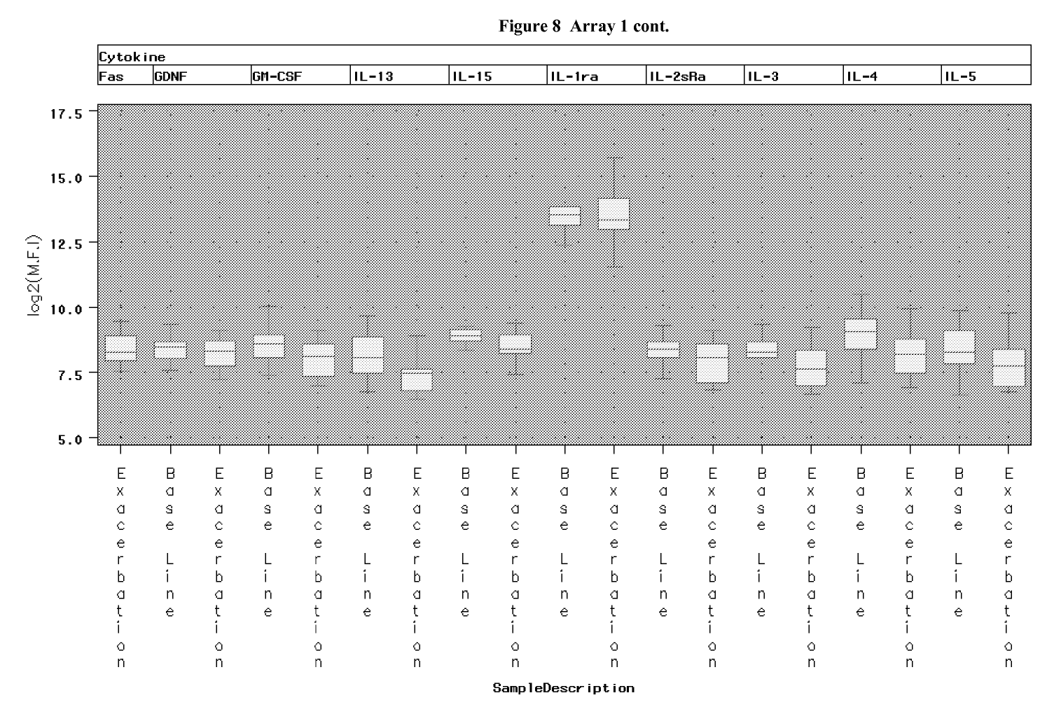

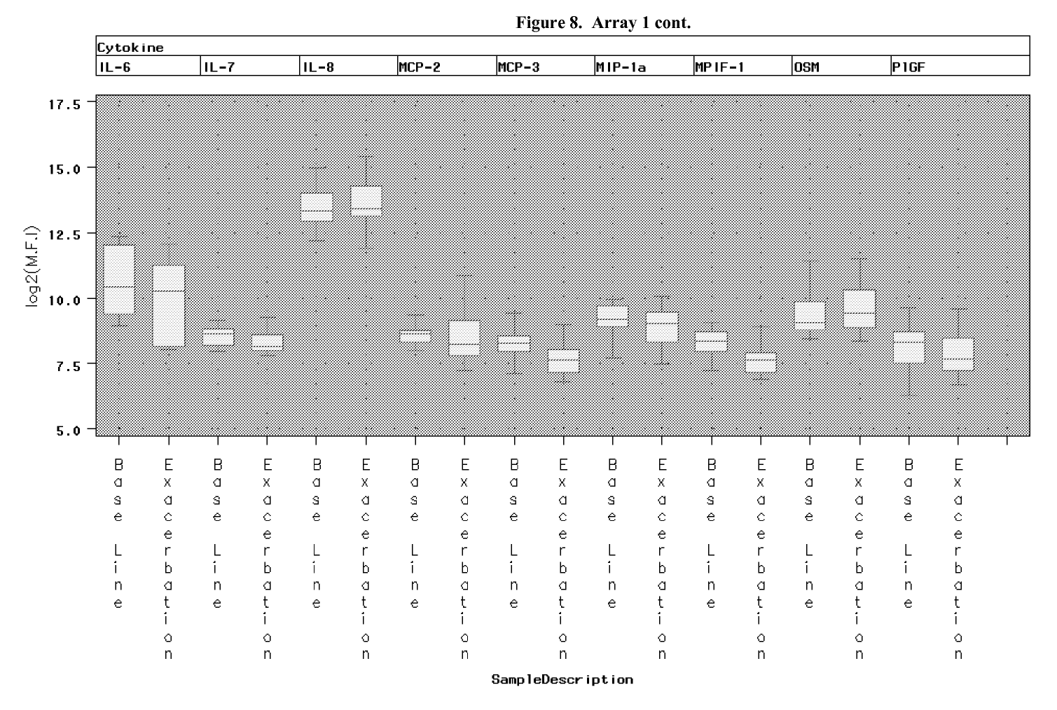

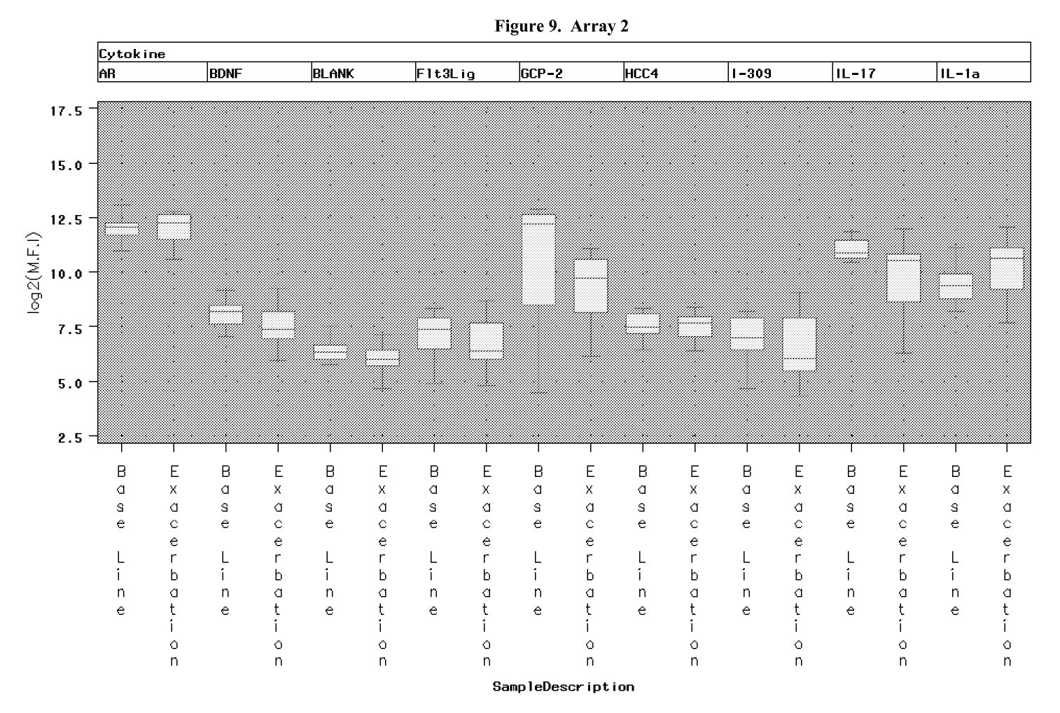

The number of analytes, expressed in baseline samples at levels higher than that of the control blank features, was an empiric part of the evaluation of the usefulness of MSI protein arrays for sputum and indicated how many analytes MSI protein microarrays would yield informative data in COPD. All analytes, except β-NGF, showed MFI (mean fluorescence intensity) values higher than the blank features on the corresponding array (FIGS. 8-11). An additional, more stringent indicator for the number of informative analytes in sputum would be the number of analytes expressed at levels >1000 MFI. Sixty-two of the 107 analytes (58%) were detected at levels >1000 MFI in baseline sputum samples (Table 14). It is important to note that the 1000 MFI cutoff is not a true limit for analyte detection and analytes expressed at lower levels may be biologically relevant. In addition, non-quiescent sputum samples (e.g., from patients with exacerbated COPD or following various treatments) may express different numbers of analytes with levels >1000 MFI. This exploratory analysis shows that a large number of analytes can be detected in the quiescent sputum samples tested in this study, indicating that sputum can be an informative sample type when tested on the MSI protein chip platform.

TABLE 14
Analytes detected at levels greater than
1000 MFI in baseline sputum samples

ALCAM
ANG
AR
CD27
CD30
CTACK
DR6
EGF
ENA-78
Eot-3
Eot2
FGF-4
FGF-9
FGF-basic
Follistatin
GCP-2
GM-CSF
GRO-g
GROb
HGF
HVEM
I-TAC
ICAM-1
IGF-II
IGF-IR
IGFBP-1
IGFBP-3
IGFBP-4
IGFBP2
IL-10rb
IL-17
IL-1a
IL-1b
IL-1ra
IL-1srII
IL-2rb
IL-4
IL-6
IL-8
L Selectin
Lymphotactin
M-CSF R
MCP-1
MCP-2
MIG
MIP-1a
MIP-1b
MIP-1d
MIP-3a
MMP7
MMP9
OSM
PARC
PECAM1
SDF-1b
sgp130
TGF-a
TIMP-2
TIMP1
TNF-R1
TRAIL R1
TRAIL R4
VEGF

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method of diagnosing chronic obstructive pulmonary disease in a patient comprising:
comparing a first concentration of prolactin in a test sample from the patient to a second concentration of prolactin in a reference range determined from one or more control samples obtained from one or more human subjects not suffering from chronic obstructive pulmonary disease; and
diagnosing chronic obstructive pulmonary disease in said patient if said first concentration of prolactin is elevated in the test sample relative to said second concentration.

2. The method of claim 1, wherein said test sample is selected from the group consisting of serum, sputum, blood, plasma, and cerebrospinal fluid.

3. The method of claim 1, wherein said one or more human subjects not suffering from chronic obstructive pulmonary disease are smokers and the method further comprises:
comparing a first concentration of at least one analyte selected from the group consisting of IGF-II and IGFBP-3 in said test sample to a second concentration of said analyte in a reference range determined from one or more control samples obtained from said human subjects; and
diagnosing chronic obstructive pulmonary disease in said patient if said first concentration of said at least one analyte is elevated in said test sample relative to said second concentrations.

4. The method of claim 1, further comprising:
comparing a first concentration of neutrophil elastase in said test sample to a second concentration of neutrophil elatase in a reference range determined from one or more control samples obtained from one or more human subjects not suffering from chronic obstructive pulmonary disease; and
diagnosing chronic obstructive pulmonary disease in said patient if said first concentration of prolactin and said first concentration of neutrophil elastase are elevated in said test sample relative to said second concentrations.

5. The method of claim 4, wherein said one or more human subjects not suffering from chronic obstructive pulmonary disease are smokers and the method further comprises:
comparing a first concentration of at least one analyte selected from the group consisting of insulin-like growth factor II (IGF-II) and insulin-like growth factor binding protein 3 (IGFBP-3), in said test sample to a second concentration of said analyte in a reference range determined from one or more control samples obtained from said human subjects; and
diagnosing chronic obstructive pulmonary disease in said patient if said first concentration of said at least one analyte is elevated in said test sample relative to said second concentrations.

6. A method of diagnosing chronic obstructive pulmonary disease in a patient comprising:
comparing a first concentration of at least one analyte in a test sample from said patient to a second concentration of the at least one analyte in a reference range determined from one or more control samples obtained from one or more human subjects not suffering from chronic obstructive pulmonary disease, wherein the at least one analyte is selected from the group consisting of matrix metalloprotease 9 (MMP-9), matrix metalloprotease 10 (MMP-10), eotaxin 2 (Eot-2), thymus and activation regulated chemokine (TARC), matrix metalloprotease 7 (MMP-7), neutrophil elastase, interleukin 8 (IL-8), macrophage migration inhibitor factor (MIF), interleukin 10 receptor (IL-10Rβ), eotaxin, matrix metalloprotease 8 (MMP-8), brain-derived neurotrophic factor (BDNF), tissue inhibitor of metalloprotease 1 (TIMP-1), amphiregulin, fibroblast growth factor 4 (FGF-4), insulin-like growth factor binding protein 4 (IGFBP-4), tumor necrosis factor receptor 1 (TNF-RI), B lymphocyte chemoattractant (BLC), cutaneous T cell attracting chemokine (CTACK), hemofiltrate CC chemokine 4 (HCC4), interleukin 12p40 (IL-12p40), monocyte chemotactic protein 1 (MCP-1), vascular endothelial growth factor (VEGF), myeloid progenitor inhibitory factor-1 (MPIF-1), hemofiltrate CC chemokine 1 (HCC1), epidermal growth factor (EGF), macrophage inhibitor protein-Ib (MIP-1b), and prolactin; and
diagnosing chronic obstructive pulmonary disease in said patient if said first concentration of said at least one analyte is elevated in said test sample relative to said second concentration.

7. The method of claim 6, wherein said one or more human subjects not suffering from chronic obstructive pulmonary disease are non-smokers.

8. The method of claim 7, wherein said at least one analyte is MMP-9.

9. The method of claim 7, wherein said at least one analyte is MMP-10.

10. The method of claim 7, wherein said at least one analyte is Eot-2.

11. The method of claim 7, wherein said at least one analyte is TARC.

12. The method of claim 7, wherein said at least one analyte is MMP-7.

13. The method of claim 7, wherein said at least one analyte is IL-8.

14. The method of claim 7, wherein said at least one analyte is MIF.

15. The method of claim 7, wherein said at least one analyte is IL-10Rβ.

16. The method of claim 7, wherein said at least one analyte is eotaxin.

17. The method of claim 7, wherein said at least one analyte is MMP-8.

18. The method of claim 7, wherein said at least one analyte is BDNF.

19. The method of claim 7, wherein said at least one analyte is TIMP-1.

20. The method of claim 7, wherein said at least one analyte is amphiregulin.

21. The method of claim 7, wherein said at least one analyte is neutrophil elastase.

22. A method of diagnosing chronic obstructive pulmonary disease in a patient comprising:
comparing a first concentration of neutrophil elastase in a test sample from said patient to a second concentration of neutrophil elastase in a reference range determined from one or more control samples obtained from one or more human subjects not suffering from chronic obstructive pulmonary disease and wherein said one or more human subjects not suffering from chronic obstructive pulmonary disease are smokers, and
diagnosing chronic obstructive pulmonary disease in said patient if said first concentration of neutrophil elastase is elevated in said test sample relative to said second concentration.

23. The method of claim 22, wherein the method further comprises
comparing a first concentration of at least one analyte selected from the group consisting of IGF-II and IGFBP-3 in said test sample to a second concentration of said analyte in a reference range determined from one or more control samples obtained from said human subjects; and
diagnosing chronic obstructive pulmonary disease in said patient if said first concentration of said at least one analyte is elevated in said test sample relative to said second concentrations.

24. A method of distinguishing exacerbator patients in chronic obstructive pulmonary disease from non-exacerbator patients, the method comprising:
comparing a first concentration of at least one analyte in a test sample from said exacerbator patient to a second concentration of said at least one analyte in a reference range determined from one or more samples obtained from one or more non-exacerbator patients suffering from chronic obstructive pulmonary disease, wherein said at least one analyte is selected from a group consisting of BLC, hepatocyte growth factor (HGF), and macrophage inhibitor protein-I delta (MIP-1 delta), and wherein said first concentration of said at least one analyte is elevated relative to said second concentration.

25. The method of claim 24, wherein said test sample is selected from the group consisting of serum sputum, blood, plasma, and cerebrospinal fluid.

26. The method of claim 24, wherein said at least one analyte is BLC.

27. The method of claim 24, wherein said at least one analyte is HGF.

28. The method of claim 24, wherein said at least one analyte is MIP-1 delta.

29. A method of diagnosing chronic obstructive pulmonary disease in a patient comprising:
assaying in a test sample from said patient a panel having two or more analytes by comparing a first concentration of each analyte in the panel to a second concentration of each analyte in said panel wherein said second concentration comprises a reference range determined from one or more control samples obtained from one or more human subjects not suffering from chronic obstructive pulmonary disease,
diagnosing chronic obstructive pulmonary disease in said patient if said first concentrations of said two or more analytes are elevated in said test sample relative to said second concentrations,
wherein said panel comprises at least one matrix metalloprotease selected from the group consisting of matrix metalloprotease 7 (MMP-7), matrix metalloprotease 8 (MMP-8), matrix metalloprotease 9 (MMP-9), and matrix metalloprotease 10 (MMP-10) and at least one analyte selected from the group consisting of Eot-2, TARC, neutrophil elastase, BDNF, IL-8, TIMP-1, and amphiregulin.

30. The method of claim 29, wherein said at least one analyte is Eot-2.

31. The method of claim 29, wherein said at least one analyte is TARC.

32. The method of claim 29, wherein said at least one analyte is neutrophil elastase.

33. The method of claim 29, wherein said at least one analyte is BDNF.

34. The method of claim 29, wherein said at least one analyte is IL-8.

35. The method of claim 29, wherein said at least one analyte is TIMP-1.

36. The method of claim 29, wherein said at least one analyte is amphiregulin.
